SEQUENCE LISTING (20 sequences)

SEQ ID NO: 1 - DNA, 19 bp, Artificial Sequence
  Description of Artificial Sequence Synthetic primer
gaaggtgaaggtcggagtc

SEQ ID NO: 2 - DNA, 23 bp, Artificial Sequence
  Description of Artificial Sequence Synthetic primer
ctggtgaaatcccggaaaaatga

SEQ ID NO: 3 - DNA, 20 bp, Artificial Sequence
  Description of Artificial Sequence Synthetic primer
atcggttgttcaatgcgtcc

SEQ ID NO: 4 - DNA, 20 bp, Artificial Sequence
  Description of Artificial Sequence Synthetic primer
cggaaagcacccgtatggag

SEQ ID NO: 5 - DNA, 21 bp, Artificial Sequence
  Description of Artificial Sequence Synthetic primer
tccagctactactcgtgtgac

SEQ ID NO: 6 - DNA, 20 bp, Artificial Sequence
  Description of Artificial Sequence Synthetic primer
actccgaggagatgaacgga

SEQ ID NO: 7 - DNA, 20 bp, Artificial Sequence
  Description of Artificial Sequence Synthetic primer
cgctactggtgtaatgatgg

SEQ ID NO: 8 - DNA, 18 bp, Artificial Sequence
  Description of Artificial Sequence Synthetic primer
tgcctggccctgtctttg

SEQ ID NO: 9 - DNA, 20 bp, Artificial Sequence
  Description of Artificial Sequence Synthetic primer
tacacgtccatcctgtacgg

SEQ ID NO: 10 - DNA, 21 bp, Artificial Sequence
  Description of Artificial Sequence Synthetic primer
gagaaagcatttgtaggcaac

SEQ ID NO: 11 - DNA, 20 bp, Artificial Sequence
  Description of Artificial Sequence Synthetic primer
gaagatggtgatgggatttc

SEQ ID NO: 12 - DNA, 22 bp, Artificial Sequence
  Description of Artificial Sequence Synthetic primer
ttgctgccaaactatcttgtga

SEQ ID NO: 13 - DNA, 23 bp, Artificial Sequence
  Description of Artificial Sequence Synthetic primer
ccttcaggatttggtacatgaca

SEQ ID NO: 14 - DNA, 20 bp, Artificial Sequence
  Description of Artificial Sequence Synthetic primer
cgtccaccacgcctacatag

SEQ ID NO: 15 - DNA, 22 bp, Artificial Sequence
  Description of Artificial Sequence Synthetic primer
ccctctgttgggaattgttctg

SEQ ID NO: 16 - DNA, 20 bp, Artificial Sequence
  Description of Artificial Sequence Synthetic primer
cttggagagcgaggtcttgg

SEQ ID NO: 17 - DNA, 20 bp, Artificial Sequence
  Description of Artificial Sequence Synthetic primer
tttgcacaagctacagcatc

SEQ ID NO: 18 - DNA, 18 bp, Artificial Sequence
  Description of Artificial Sequence Synthetic primer
cagctccagcatgagtgc

SEQ ID NO: 19 - DNA, 21 bp, Artificial Sequence
  Description of Artificial Sequence Synthetic primer
ctcgctctcattcacgtctgg

SEQ ID NO: 20 - DNA, 20 bp, Artificial Sequence
  Description of Artificial Sequence Synthetic primer
atctcccaacaaactggatg

(19) United States
(12) Patent Application Publication
(10) Pub. No.: US 2019/0300860 A1
(43) Pub. Date: Oct. 3, 2019

(54) Innervated Artificial Intestine Compositions

(71) Applicant: Trustees of Tufts College, Medford, MA (US)

(72) Inventors: David L. Kaplan, Concord, MA (US); Ying Chen, Winchester, MA (US); Wenda Zhou, Cambridge, MA (US); Dana Cairns, Somerville, MA (US); Fiorenzo G. Omenetto, Lexington, MA (US); Eleana Manousiouthakis, Los Angeles, CA (US)

(21) Appl. No.: 16/317,456

(22) PCT Filed: Jul. 11, 2017
(86) PCT No.: PCT/US17/41550

§ 371 Date: Jan. 11, 2019

Related U.S. Application Data
(60) Provisional application No. 62/360,837, filed on Jul. 11, 2016.

(51) Int. Cl. C12N 5/071; C12N 5/00; C12N 5/077

(52) U.S. Cl. CPC C12N 5/0697; C12N 5/0062; C12N 5/0613; C12N 2533/50; C12N 5/0656; C12N 2503/04; C12N 2513/00; C12N 5/0679

(57) ABSTRACT

The present invention provides, among other things, compositions including a plurality of enterocytes, a plurality of fibroblasts, a plurality of Goblet cells, a plurality of Paneth cells, a plurality of enteroendocrine cells, and a silk fibroin scaffold, wherein the composition exhibits one or more of tight junction formation, microvilli polarization, digestive enzyme secretion, and low oxygen tension and methods of making and using the same. In some embodiments, the composition exhibits one or more of tight junction maintenance, maintenance of microvilli polarization, digestive enzyme secretion, and low oxygen tension for at least 10 days.

### BACKGROUND

The intestine is central to human health and the enteric nervous system functions as the main component of the autonomic nervous system. As such, intestinal neuromodulation impacts many aspects of human health and an improved understanding of such a system would have major implications improving the health of patients worldwide. The enteric nervous system (ENS) has been termed the ‘second brain’ due to its central role in autonomy, functional control and impact in the human body. Current options to assess intestine neuromodulatory functions are primarily limited to animal studies, cell culture studies, organoids and related systems, all of which have inherent limitations.

### SUMMARY

In some embodiments, the present invention provides compositions including a plurality of enterocytes, a plurality of fibroblasts, a plurality of Goblet cells, a plurality of Paneth cells, a plurality of enteroendocrine cells, and a silk fibroin scaffold, wherein the composition exhibits one or more of tight junction formation, microvilli polarization, digestive enzyme secretion, and low oxygen tension. In some embodiments, the composition exhibits one or more of tight junction maintenance, maintenance of microvilli polarization, digestive enzyme secretion, and low oxygen tension for at least 10 days (e.g., at least 11 days, 15 days, 20 days, 25 days, 30 days, 60 days, 90 days, or 180 days). Unless otherwise specified, the terms “composition”, “provided composition”, and “intestine-like composition” are used interchangeably herein.

In some embodiments, the present invention provides methods including the steps of providing a silk fibroin scaffold, associating a plurality of fibroblasts with the silk fibroin scaffold, associating a plurality of intestinal stem cells with the silk fibroin scaffold, differentiating the plurality of intestinal stem cells into two or more of enterocytes, Goblet cells, Paneth cells, and enteroendocrine cells to form an intestine-like composition. In some embodiments, intestinal stem cells do not include totipotent stem cells. In some embodiments, intestinal stem cells do not include pluripotent stem cells. In some embodiments, intestinal stem cells do include multipotent stem cells (i.e., cells capable of differentiating into enterocytes, Goblet cells, Paneth cells, and/or enteroendocrine cells).

In some embodiments, provided compositions secrete one or more digestive enzymes. By way of specific example, in some embodiments, the digestive enzyme secretion is or comprises secretion of one or more of alkaline phosphatase, secretin, cholecystokinin, maltase, lactase, gastric inhibitory peptide, motilin, somatostatin, erepsin, and sucrase.

In some embodiments, provided compositions may exist in or include portions that exhibit a state low oxygen tension. In some embodiments, the low oxygen tension means less than 5% pO₂. In some embodiments, the low oxygen tension means less than 2% pO₂. In some embodiments, provided compositions exhibit a depth-graded oxygen profile, for example, in a luminal direction. By way of specific example, in some embodiments wherein a silk fibroin scaffold includes a hollow channel and thus a lumen, when such provided compositions are oriented vertically, in the scaffold lumen, a region of microaerobic conditions (pO2 between 5% and 1%) may be detected at depths ranging from 2 to 5 mm into the scaffold lumen; and a nanaerobic region (pO2 ˜1%) may be detected at the depth of 5 to 6 mm.

As discussed herein, innervation is a critical part of intestinal health and function. Accordingly, in several embodiments, provided compositions further include a plurality of nervous system cells. In some embodiments, the nervous system cells are human nervous system cells. In some embodiments, the nervous system cells are or comprise afferent nerve cells. In some embodiments, the nervous system cells are or comprise efferent nerve cells. In some embodiments, the nervous system cells comprise glial cells. In some embodiments, at least some of the plurality of nervous system cells provide functional innervation to at least some of the enterocytes, Paneth cells, enteroendocrine cells, and/or Goblet cells.

In some embodiments, the nervous system cells comprise neuronal nitric oxide synthase (nNOS)-expressing neurons. Prior to the present invention, the only known way to achieve nNOS expression in an in vitro system was through direct transplant into mice. As such, this represents the first time development of nNOS expressing neurons has been observed in an entirely in vitro system (e.g., an entirely in vitro system including all human cells).

In some embodiments, provided compositions are capable of initiating an antimicrobial response (e.g., in response to a microbe or portion thereof). In some embodiments, an antimicrobial response is or comprises upregulated gene and/or protein expression of one or more of lymphocyte antigen 96 (LY96), toll-like receptor-2 (TLR2), toll-like receptor-4 (TLR4), toll-like receptor-5 (TLR5), toll-like receptor-6 (TLR6), c-reactive protein (CRP), deleted in malignant brain tumors-1 (DMBT1), interferon regulatory factor-7 (IRF7), z-DNA-binding protein 1 (ZBP1), chemokine (C-C motif) ligand 3 (CCL3), C-X-C motif chemokine 1 (CXCL1), C-X-C motif chemokine 2 (CXCL2), interleukin-12 subunit alpha (IL12A), interleukin-12 subunit beta (IL12B), interleukin 1 beta (IL1B), interleukin 6 (IL6), myeloid differentiation primary response gene 88 (MYD88), nucleotide-binding oligomerization domain-containing protein 1 (NOD1), nucleotide-binding oligomerization domain-containing protein 2 (NOD2), Ras-related C3 botulinum toxin substrate 1 (RAC1), p65 (RELA), tumor necrosis factor (TNF), bactericidal permeability-increasing protein (BPI), cathelicidin (CAMP), cathepsin G (CTSG), lysozyme (LYZ), myeloperoxidase (MPO), secretory leukocyte protease inhibitor (SLPI), mitogen-activated protein kinase kinase 1 (MAP2K1), mitogen-activated protein kinase 1 (MAPK1), mitogen-activated protein kinase 8 (MAPK8), JUN, killer cell immunoglobulin-like receptor subunit a (NKB1A), caspace 1 (CASP1), and apoptosis-associated speck-like protein containing a CARD (PYCARD).

In some embodiments, provided compositions do not comprise any immortalized cells. In some embodiments, provided compositions do not comprise adenocarcinoma-based cells. In some embodiments, all of the cells present in the composition are human cells. In some embodiments, at least one of the plurality of Enterocytes, Goblet cells, Paneth cells, and enteroendocrine cells originated from a patient.

Any of a variety of silk fibroin scaffolds may be used in accordance with various embodiments. For example, in some embodiments, the silk fibroin scaffold is a film, a sponge, a tube, a mat, a gel, or any of the foregoing including a hollow channel. In some embodiments, a silk fibroin scaffold is porous. In some embodiments, provided compositions may comprise at least one additional silk fibroin scaffold (i.e., a second, third, fourth, etc silk fibroin scaffold).

In some embodiments, provided compositions may further comprise an electrical device that is functionally connected to at least some of the plurality of nervous system cells. In some embodiments, the electrical device comprises at least one electrode. In some embodiments, the electrical device comprises silk fibroin.

In some embodiments, provided compositions may be used in analytical methods (e.g., methods to characterize the response of one or more intestinal cell types to a therapeutic agent). In some embodiments, the present invention also provides methods including the steps of providing a composition including a plurality of enterocytes, a plurality of fibroblasts, a plurality of Goblet cells, a plurality of Paneth cells, a plurality of enteroendocrine cells, and a silk fibroin scaffold, wherein the composition exhibits one or more of tight junction formation, microvilli polarization, digestive enzyme secretion, and low oxygen tension, exposing the composition to one or more therapeutic agents, and characterizing the response of one or more of the enterocytes, Goblet cells, Paneth cells, and enteroendocrine cells to the one or more therapeutic agents. In some embodiments, at least some of the enterocytes, Goblet cells, Paneth cells, and/or enteroendocrine cells exhibit one or more pathologic abnormalities as compared to similar cells from a healthy individual prior to the exposing step. In some embodiments, the one or more pathologic abnormalities is indicative of, or correlated to, the presence of a disease. In some embodiments, the disease is selected from the group consisting of inflammatory bowel syndrome, Celiac Disease, Crohn's disease, intestinal cancer, intestinal ulcer, ulcerative colitis, and diverticulitis.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1. Fabrication process. The bioreactor fabricated from two layer walls, a PDMS made outside wall (orange) and ECOFLEX® made inner wall (light blue). There was an airtight chamber between the inner wall and the outside walls. Two adaptors (dark blue) were located on the center of basement and the lid separately. The intestinal tissue (green) was fit between those two adaptors.

FIG. 2. Function process. The two adaptors provide a channel to perfuse the tissue. The medium (red) was pump from the one adaptor and perfuse through the tissue and then flow through the other adaptor. By change the air pressure in the air chamber, the highly flexible ECOFLEX® were physically deform intestinal tissue to provide peristaltic associated mechanically stimulation.

FIG. 3. Design of a bioreactor system with oxygen control system. The bioreactor comprised a perfusion circuit (red) and a peristaltic motion system. The oxygen tensions in the medium (pO₂) were reduced to the either microaerophilic (˜5%) level, anaerobic (˜1%) level or a fully anaerobic level (<0.1%) in the luminal circuit, by purging the medium with different mixtures of O₂/CO₂/N₂ or H₂/CO₂/N₂.

FIG. 4. Two weeks after incubation. SEM and confocal image (DAPI/Z0-1) of epithelium layer of the scaffold at 2 weeks after incubation. Panels A and B show groups without mechanical stimulation. Panels C and D show groups with mechanical stimulation at 2 cycle/min. Panels E and F show groups with mechanical stimulation at 5 cycle/min.

FIG. 5. Five weeks after incubation. SEM and confocal image (DAPI/Z0-1) of epithelium layer of the scaffold at 5 weeks after incubation. Panels A, B, and C show mechanical stimulation group and panels D, E, and F show control group (without mechanical stimulation).

FIG. 6. Panel A (subpanels a-d) ZO-1 staining of human small intestinal primary cells seeded on 3D silk scaffolds up to 10 days. Subpanels e-h show various intestinal epithelial cell subtypes in the epithelium, which includes enterocytes (e), Goblet cells (f), Paneth cells (g), and enteroendocrine cells (h). Panel B shows Confocal microscopy of immunostained ZO-1 (subpanel a), Villin (subpanel b), Muc2 (subpanel c) of primary large intestinal epithelial cells cultured on 3D silk scaffolds 7d post cell seeding. Scale bar=50 μm. Panel C shows (subpanel a) Confocal images showed that after culture for 3 days in differentiation medium cells isolated from human intestinal enteroids expressed F-actin (red) and ZO-1 (green). Subpanels b-e show four major subpopulations of intestinal epithelial cells observed in the differentiated epithelium. Scale bar=25 μm. Panel D shows confocal images show large intestinal primary cells isolated from colonoids highly expressed Muc-2 (red) and ZO-1 (green). Scale bar=30 μm.

FIG. 7: Human Intestine in vitro. Panels A-C show exemplary design of a 3D silk scaffold platform. Panel D shows representative images of immunostaining of F-actin, ZO-1, MUC-2, and ALP stain on epithelium grown on 3D silk scaffold lumens (upper panel: non-patterned, lower panel: patterned) at day 10. Scale bars=1 mm, 60 um, 200 μm, 200 μm.

FIG. 8: (LEFT) Conformal Electrodes on the Brain—panel a shows a schematic of clinical use of a representative device in an ultrathin mesh geometry with a dissolvable silk support. Panel b shows a picture of an electrode array conforming to a circular surface as silk is dissolved. Panels c and d show pictures of an electrode array on a cat brain. (RIGHT) Silk wireless antennas for diagnostics on teeth and bacterial detection. Preliminary results using conformal sensors on biological surfaces for wireless bacterial detection. Panel (a) shows a gold wireless antenna is manufactured onto silk. Panel (b) shows the device can be conformally transferred on a variety of surfaces (such as a tooth). Panel (c) shows a magnified schematic of an exemplary sensing element. Panel (d) shows an exemplary actual provided silk-device, panel (e) shows a sensor transferred on to chicken skin and panel (f) shows a sensor transferred on to tooth enamel.

FIG. 9 Overview of the cell seeding strategy for HIE-derived 3D intestinal constructs. Panels a and b show HIEs isolated from human patients are cultured in the Matrigel. Panel c shows HIEs were enzymatically digested to obtain Singlet/doublet cells. Panel d shows HIE-derived cells were seeded onto the luminal surface of a 3D tubular silk scaffold, while H-InMyoFibs were delivered into the spongy silk scaffold bulk. The constructs were cultured in differentiation medium for at least 3 days to induced intestinal epithelial differentiation. Panel e shows SEM photographs of microvilli brush border formation at the apical cell surface. Scale bar, 10 μm. Panel f shows highly organized ZO-1 chicken wire pattern staining in differentiated HIE-derived epithelium on 3D scaffolds. Scale bar, 15 μm. Panel g shows ALP staining on the epithelial cells were observed on the epithelium. Scale bar, 250 μm.

FIG. 10 shows exemplary graphs of gene expression levels of four intestinal epithelial cell markers, including SI (panel a), Muc2 (panel b), Lysozyme (panel c) and ChgA (panel d), functional epithelium markers, including ZO-1 (panel e), Villi (panel f), and ALP (panel g), and an intestinal stem cell marker, Lgr5 (panel h) were evaluated by quantitative reverse transcription-polymerase chain reaction (qRT-PCR) overtime in cultures. Data is presented as mean±SEM, n=5 in each group, p<0.001.

FIG. 11 shows exemplary photographs of the density of microvilli and formation of continuous brush borders across cells in exemplary HIE-derived compositions as compared to hInEpiC-derived and cell line-derived compositions. Panels A and B show exemplary SEM images of microvilli on human intestinal enteroids grown on 3D scaffolds from day 3. Panels C and D show exemplary SEM images of microvilli on primary human small intestinal epithelial cells grown on 3D scaffolds from day 5. Panels E and F show exemplary SEM images on microvilli of intestinal cell lines grown on 3D scaffolds from day 5.

FIG. 12 shows the differentiation of 3D intestinal epithelia cell subtypes. Panels a and b show a schematic of the general seeding cell seeding strategy for hInEpiC-derived and cell line-derived 3D intestinal constructs. Panels c-n show immunohistological stainings of SI (sucrose-isomaltase, panels c, g, k), MUC-2 (Mucin 2, panels d, h, 1), Lysozyme (panels e, i, m) and ChgA (Chromogranin A, panels f, g, n) showed the location of enterocytes, Goblet cells, Paneth cells, and enteroendocrine cells in differentiated HIE-derived, hInEpiC-derived cell line-derived epithelia. Scale bar, 25 μm. Panel 0 shows the fold-change in mRNA expression of SI, Muc-2, Lysozyme, and ChgA over a time period of up to 3 weeks as compared with cell line-derived 3D constructs at day 1 post cell seeding.

FIG. 13 shows schematics of an exemplary composition as well as graphs of the oxygen concentration profiles of HIE-derived (panel a), hInEpiC-derive (panel b), and cell line-derived (panel c) tissues 3D tissues were measured using an oxygen meter.

FIG. 14 shows Human Antibacterial Response RT2 Profiler™ PCR arrays (panels a-c). Heat-map comparison of 84 antibacterial genes in HIE-derived (panel a), hInEpiC-derived (panel b), and cell line-derived (panel c) epithelia after exposure to E. coli. for 4 hours. Genes were displayed for fold-change variation in respect to their uninfected control groups and colored by their normalized expression value (red: high expression; green: low expression) as shown in panels d-f via scatter plot charts. Genes upregulated with fold change greater than 4 and are showed as red dots; genes with fold change less than 4 are showed as green dots; unmodulated genes are showed as black. Panel g shows a heatmap detail displayed all upregulated genes for HIE-derived, hInEpiC-derived, and cell line-derived epithelia after E. coli infection.

FIG. 15 shows exemplary photographs of hiNSCs, hiNSC colonies that were dissociated into single-cell suspension and injected into the lumen of the developing neural tube at day 3 (D3). Intestinal samples were collected at D14 in order to assess migration of nerve cells. Image shows series of confocal images of whole mount immunostained intestines with anti-nuclei (red) to stain cells of human origin and TUJ1 (green). Scale 75 μm.

FIG. 16 shows a schematic diagram of the timeline of seeding for certain exemplary provided compositions comprising a simulated Enteric Nervous System.

FIG. 17 shows photographs of exemplary provided compositions at day 30 (panels A, B) and day 45 (panels C, D) seeded with hiNSCs and epithelial cells (Caco2 and HT29-MTX) stained with calcein-AM viability dye (green). Images show focal planes to represent live cells seen in the bulk of the scaffold (panels A, C) as well as the lumen of the scaffold (panels B, D) for each time point. Scale bar 500 μm.

FIG. 18 shows a graph of Alamar Blue fold change data for certain provided compositions as compared to cell free scaffolds over approximately three weeks of culture. hiNSC=hiNSC only scaffold, INT=intestinal cells only scaffold, CC=co-culture scaffold with both intestinal and hiNSCs.

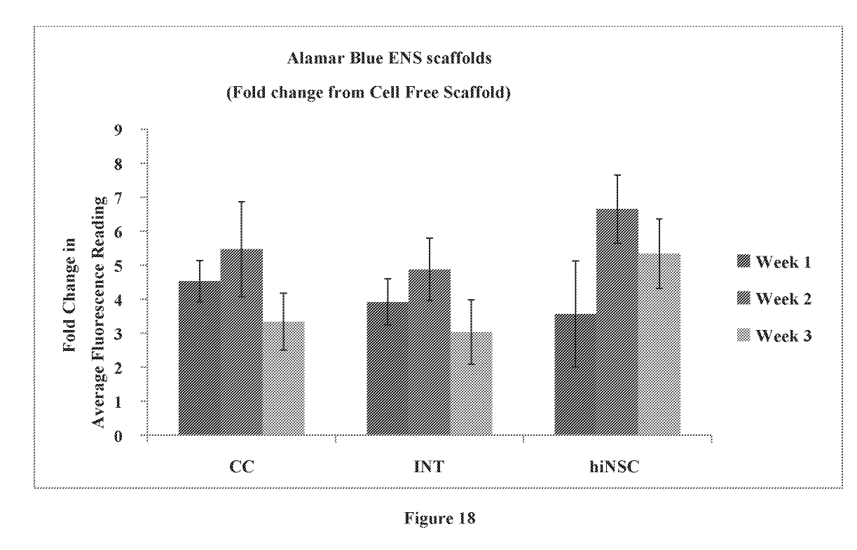

FIG. 19 shows an exemplary schematic of certain provided compositions (leftmost panel) and cell seeding thereof. Scale bar=2 mm. Regarding the rightmost panel, subpanel A shows an image of immunostained sections of hiNSCs infiltrating toward scaffold lumen. Sub-panels A and B show staining for the pan neuronal marker beta III tubulin TUJ1 (green), tight junction marker ZO-1(red) indicates epithelium, and nuclear stain DAPI (blue) on 3D scaffold lumens at week 3. Sub-panels C and D show staining for TuJ1 (red), neuronal NOS inhibitory neuron nNOS (green), and DAPI (blue) on 3D scaffold lumens at week 3.

### Definitions

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

“About”: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Comparable”: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” and “attached,” when used with respect to two or more moieties, means that the moieties are physically connected with one another, either directly or via one or more additional moieties that serves as a linking agent, to form a structure that is sufficiently stable so that the moieties remain physically connected under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically connected.

“Encapsulated”: The term “encapsulated” is used herein to refer to substances that are substantially completely surrounded by another material.

“Functional”: As used herein, a “functional” biological molecule is a biological molecule in a form in which it exhibits a property and/or activity by which it is characterized. A biological molecule may have two functions (i.e., bi-functional) or many functions (i.e., multifunctional).

“High Molecular Weight Polymer”: As used herein, the term “high molecular weight polymer” refers to polymers and/or polymer solutions comprised of polymers (e.g., protein polymers, such as silk) having molecular weights of at least about 200 kDa, and wherein no more than 30% of the silk fibroin has a molecular weight of less than 100 kDa. In some embodiments, high molecular weight polymers and/or polymer solutions have an average molecular weight of at least about 100 kDa or more, including, e.g., at least about 150 kDa, at least about 200 kDa, at least about 250 kDa, at least about 300 kDa, at least about 350 kDa or more. In some embodiments, high molecular weight polymers have a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa.

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Low Molecular Weight Polymer”: As used herein, the term “low molecular weight polymer” refers to polymers and/or polymer solutions, such as silk, comprised of polymers (e.g., protein polymers) having molecular weights within the range of about 3 kDa-about 200 kDa. In some embodiments, low molecular weight polymers (e.g., protein polymers) have molecular weights within a range between a lower bound (e.g., about 10 kDa, about 20 kDa, about 30 kDa, about 40 kDa, about 50 kDa, about 60 kDa, or more) and an upper bound (e.g., about 200 kDa, about 175 kDa, about 150 kDa, about 125 kDa, about 100 kDa, or less). In some embodiments, low molecular weight polymers (e.g., protein polymers such as silk) are substantially free of polymers having a molecular weight above about 200 kD. In some embodiments, the highest molecular weight polymers in provided hydrogels are less than about 100-about 200 kD (e.g., less than about 200 kD, less than about 175 kD, less than about 150 kD, less than about 125 kD, less than about 100 kD, etc). In some embodiments, a low molecular weight polymer and/or polymer solution can comprise a population of polymer fragments having a range of molecular weights, characterized in that: no more than 15% of the total moles of polymer fragments in the population has a molecular weight exceeding 200 kDa, and at least 50% of the total moles of the silk fibroin fragments in the population has a molecular weight within a specified range, wherein the specified range is between about 3 kDa and about 120 kDa or between about 5 kDa and about 125 kDa.

“Matrix”: As used herein, the term “matrix” refers to a biomaterial comprising silk fibroin or collagen or combinations of these two as well as with ECM components, on or in which cells will grow.

“Nucleic acid”: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., and atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, www.cco.caltech.edu/\tilde{ }dadgrp/Unnatstruct.gif, which displays structures of non-natural amino acids that have been successfully incorporated into functional ion channels) and/or amino acid analogs as are known in the art may alternatively be employed). For example, a polypeptide can be a protein. In some embodiments, one or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Porosity”: The term “porosity” as used herein, refers to a measure of void spaces in a material and is a fraction of volume of voids over the total volume, as a percentage between 0 and 100%. A determination of porosity is known to a skilled artisan using standardized techniques, for example mercury porosimetry and gas adsorption (e.g., nitrogen adsorption).

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Scaffold”: As used herein, the term “scaffold” refer to a three dimensional architecture that is generated from a matrix. Non-limiting examples include tubes, films, fibers, foams, gels, ring shaped structures, porous versions of any of the foregoing, and combinations thereof. In some embodiments, a scaffold may comprise a hollow channel running either along its length and/or through a portion of the scaffold.

“Solution”: As used herein, the term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Stable”: The term “stable,” when applied to compositions herein, means that the compositions maintain one or more aspects of their physical structure and/or activity over a period of time under a designated set of conditions. In some embodiments, a period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Sustained release”: The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

### DETAILED DESCRIPTION OF CERTAIN EMBODIMENTS

Prior to the present invention, there was no in vitro intestinal model that was able to accurately mimic the physiology and function of the enteric nervous system in native intestine. As such, previously known intestinal models were not physiologically relevant, and were limited in their applicability. In contrast, various embodiments of the present invention are capable of use as a closed loop neuromodulation system sufficient to emulate endogenous neural signaling. Accordingly, several embodiments are capable of being used as disease models and/or in transplant of tissue to a subject in need, among other uses.

In some embodiments, the present invention provides methods and compositions including new intestine-like compositions which include silk fibroin. In some embodiments, provided methods and compositions include primary human cells and/or intestinal organoids, rather than the immortalized cells or cell lines used in previous systems. In some embodiments, provided compositions show innervation, for example, through the use of human induced neural stem cells (hiNSCs), allowing for study of, inter alia, gut-brain signaling, enteric nervous system signaling and potential therapeutic applications thereof. In some embodiments, provided compositions comprise functional neurons (e.g., derived from hiHSCs) which are able to approximate the ganglionated submucosal plexuses housed in the intestinal wall within a silk scaffold.

In some embodiments, provided methods and compositions are able to support perfusion and/or mechanical stimulation (e.g., peristalsis), thus allowing for lumen contents to transit through provided compositions.

In some embodiments, the present invention provides compositions including a plurality of enterocytes, a plurality of fibroblasts, a plurality of Goblet cells, a plurality of Paneth cells, a plurality of enteroendocrine cells, and a silk fibroin scaffold, wherein the composition exhibits one or more of tight junction formation, microvilli polarization, digestive enzyme secretion, and low oxygen tension. In some embodiments, the composition exhibits one or more of tight junction maintenance, maintenance of microvilli polarization, digestive enzyme secretion, and low oxygen tension for at least 10 days (e.g., at least 11 days, 15 days, 20 days, 25 days, 30 days, 60 days, 90 days, or 180 days). Unless otherwise specified, the terms “composition”, “provided composition”, and “intestine-like composition” are used interchangeably herein.

Intestinal Tissue

The human small intestine is a highly complex hollow organ located at the upper part of the intestinal tract. It is comprised of an intestinal epithelium, lamina propria, submucosa, muscularis mucosa, and serosa. The small intestinal epithelium is the innermost layer featuring two topographic structures, the villi (luminal protrusions) and crypts (luminal invaginations), on the top of which trillions of commensal microbes reside. The epithelium covering the villi encompasses at least four major cell populations: absorptive enterocyte cells, mucus-producing Goblet cells, hormone-secreting enteroendocrine cells (EECs), and antimicrobial peptide secreting Paneth cells in the crypt. All intestinal epithelial cell types are derived from proliferative crypt regions containing undifferentiated intestinal stem cells (ISCs) that self-renew to maintain stem cell populations which are identified by the specific expression of leucine rich repeat containing G protein-coupled receptor 5 gene (Lgr5). These differentiated epithelial cells enable the small intestine to perform two major physiological functions: efficient absorbance of nutrients and water from ingested food and establishment of a dynamic physical and biochemical barrier against external toxins and invading enteric pathogens. Loss of either of these functions is associated with the initiation and propagation of several intestine diseases, such as bacterial, viral, and parasitic infections, and inflammatory bowel diseases, which affect millions of people worldwide. To develop effective solutions to this worldwide problem, animal models are utilized for studies related to its causes and treatments, however, costly facilities and lack of correlations to human physiological responses limit the relevance of these animal models. This disconnect has limited the development of effective treatments to combat many of these infectious diseases, leaving large populations around the world susceptible. Tissue engineering approaches offer an alternative strategy to recapitulate human intestinal structure and function in vitro, which circumvent the limitations of animal models and provide new experimental systems with which detailed study of disease and interventions can be pursued in a more effective manner.

In the past decade, there have been many attempts to recreate in vitro bioengineered intestine-like tissue models for the study of intestinal diseases and for the development of new therapies. However, previously existing in vitro models of the human intestine rely on cultures of intestinal epithelial cell monolayers on cell culture platforms to mimic the human small intestine microenvironment. These culture platforms may be two-dimensional (2D) or three-dimensional (3D) and typically include flattened or ridged 2D substrates, microfabricated substrates, microfluidic chips, hollow fiber bioreactors, or biomaterial scaffolds. The major pitfall of the abovementioned intestine models is the use of heterogeneous human colonic adenocarcinoma cell lines, such as Caco-2 and HT-29. Cell lines are not representative of native intestinal tissue in many ways. For instance, each cell line only comprises one single cell population and fails to recapitulate the cell diversity in normal intestinal epithelium. Furthermore, the genotype of the subclones of these cell lines, especially Caco-2 cells, tends to change with increasing passage numbers or with differing culture conditions, yielding at best, inconsistent drug screening and host-pathogen interaction data. A s a result, the pharmaceutical industry, which uses cell line-derived intestinal systems for drug testing purposes, suffers high attrition rates, with less than 10% of clinical drug candidates making it to phase I testing and entering the market. In an attempt to overcome the limitations of cell lines, tissue engineers have adopted primary human small intestinal epithelial cells (hInEpiCs) which are isolated directly from native intestinal tissues for the in vitro establishment of a more physiologically relevant human small intestinal epithelium. However, hInEpiCs are difficult to isolate, remain viable for only several days and readily lose their phenotype in culture, hampering their widespread application in tissue engineering. Therefore, an alternative non-transformed epithelial cell source is needed to model a physiological 3D human intestine, and is provided in the present invention.

In some embodiments, provided compositions comprise or are created at least in part through the use of human small intestinal enteroids (HIEs). HIEs are LGR5-positive intestinal stem cells generated ex vivo from small intestinal crypt samples (endoscopic biopsies or surgical tissues) of individuals consenting to tissue donation for research. Compared to cancer cell lines and hInEpiCs, HIEs have at least two major advantages. First, HIEs, when cultured under the correct conditions, can self-renew, expand indefinitely and differentiate into all cell types of the intestinal epithelium. Secondly, HIEs are patient-specific, which may allow investigation of personalized therapeutics. Each enteorid has a micro-scaled enclosed lumen with apical cell surfaces facing the lumen and basal surfaces exposed to the Matrigel. For a long time, accessing the lumen of enteroids and inducing appropriate luminal stimulations or bacterial infections has been difficult, limiting their use in intestinal tissue modeling and disease studies.

Tight Junction Formation

In some embodiments, provided compositions include the formation of one or more tight junctions. Tight junctions, as used herein, refers to the formation of closely associated areas between two or more cells wherein the membranes of those cells form an impermeable or semi-permeable barrier to one or more substances (e.g., to microbial pathogens, toxic substances, ions, allergens, etc). In some embodiments, a tight junction will prevent or substantially prevent the passage of a fluid, ions and molecules. Epithelial tight junctions (TJs) maintain the intestinal barrier while regulating permeability of ions, nutrients, and water. The epithelial pemeability can be measured in terms of the transepithelial electrical resistance (TEER) value. TEER values of a fully differentiated intestinal epithelial monolayer range between 200-1500 Ω·cm2. The permeability can also be assessed by using permeability markers, including FITC-dextran, ovalbumin, polyethylene glycol and lactulose/mannitol. Tight junctions, as used herein, refers to the formation of closely associated areas between two or more cells wherein the membranes of those cells form an impermeable or semi-permeable barrier to one or more substances (e.g., to microbial pathogens, toxic substances, ions, allergens, etc). In some embodiments, a tight junction will prevent or substantially prevent the passage of a fluid.

Microvilli Polarization

Microvilli are finger-like projections that extend from enterocytes of the intestinal epithelium. In accordance with various embodiments, some provided compositions exhibit microvilli polarization, which may include formation of a brush border. Microvilli greatly increase the intestinal surface area, and are known to be important for nutrient absorption, mucus secretion, digestive enzyme secretion, and cellular adhesion. In vivo, microvilli also typically include a glycocalyx coating which itself comprises glycoproteins.

Digestive Enzyme Secretion

In accordance with various embodiments, provided compositions may secrete one or more digestive enzymes. By way of specific example, in some embodiments, the digestive enzyme secretion is or comprises secretion of one or more of alkaline phosphatase, secretin, cholecystokinin (CCK), maltase, lactase, gastric inhibitory peptide, motilin, somatostatin, erepsin, and sucrose, glucagon-like peptide-1, glucagon-like peptide-2, pancreatic peptide YY₃₋₃₆, neurotensin, and neurotransmitters such as serotonin and histamine. In some embodiments, one or more digestive enzymes are secreted by enteroendocrine cells, as further described below.

Low Oxygen Tension

Low oxygen tension is critical for intestinal tissue function, as it is required for maintenance of a healthy gut microbial community. Prior to the present invention, in vitro generation and dynamic control of oxygen gradients mimicking in vivo intestinal tissue, which vary from the anaerobic lumen across the epithelium into the highly vascularized sub-epithelium, has been a challenge for bioengineering and tissue regeneration. Accordingly, one of the advantages of various embodiments is that some provided compositions are able to exhibit a low oxygen tension.

In some embodiments, provided compositions may exist in or include portions that exhibit a state low oxygen tension. In some embodiments, the low oxygen tension means less than 5% pO₂. In some embodiments, the low oxygen tension means less than 2% pO₂. In some embodiments, provided compositions which comprise a lumen exhibit a depth-graded oxygen profile, for example, in a luminal direction. By way of specific example, for non-patterned scaffold lumens, in some embodiments wherein a silk fibroin scaffold includes a hollow channel and thus a lumen, when such provided compositions are oriented vertically, in the scaffold lumen, a region of microaerobic conditions (pO2 between 5% and 1%) may be detected at depths ranging from 2 to 5 mm into the scaffold lumen; and a nanaerobic region (pO2 ˜1%) may be detected at the depth of 5 to 6 mm. By way of additional specific example, for pattened-lumens, a highly oxygen-deficient, anaerobic condition (pO₂<0.1%) was found at a depth of 2 to 6 mm in the lumens.

Enterocytes

Enterocytes are absorptive intestinal cells found in the small intestine. Typically, enterocytes include a surface coating (called a glycocalyx) which includes, inter alia, digestive enzymes. Enterocytes are known to have a variety of functions including, but not limited to uptake of a variety of materials including ions, water, sugars, lipids, protein and other amino acids, and vitamins; resorption of unconjugated bile salts; and secretion of immunoglobulins.

Fibroblasts

Fibroblasts are cells are a stromal cell that is known to synthesize collagen and other components of the extracellular matrix including glycoproteins and glycosaminoglycans. In vivo, intestinal myofibroblasts are fibroblasts that reside in proximity to epithelial cells and provide nutrition and support. It is also known the fibroblasts play a role in supporting the growth and differentiation of intestinal epithelium. Further, intestinal myofibroblasts are known to be important in initiating certain antimicrobial reactions in the body (e.g., inflammation).

Goblet Cells

Goblet cells are epithelial cells whose primary functions is to secrete mucus. Goblet cells in general are highly polarized with the nucleus at one end and a large number of secretory granules at the other. The presence of the mucus containing granules are responsible for the shape form which these cells derive their name. Goblet cells may be found throughout the intestinal epithelia and in both the large and small intestines. Goblet cell function is important to intestinal health at least because mucus is critical in lubricating the surface of the intestine and protecting the tissue form insult. In vivo, the intestinal mucus layer in humans is known to be approximately 200 μm thick, though the thickness can vary somewhat in humans and may be less in other species.

Paneth Cells

Paneth cells are another type of intestinal epithelial cell that is largely found in the small intestine. The primary role of Paneth cells is in antimicrobial and other defense of tissue. Specifically, Paneth cells synthesize and secrete significant quantities of several antimicrobial proteins and peptides including at least defensins, lysozyme, tumor necrosis factor-alpha (TNFα), and phospholipase A2. In addition, it is known that Paneth cells are important in maintaining the capacity for epithelial cell renewal.

Enteroendocrine Cells

Enteroendocrine cells (“EECs”) are the most numerous endocrine cell type in the body and are known to secrete a variety of hormones and peptides in response to stimuli or various types. At least because of their role in signal transduction, EECs are thought to form an enteric endocrine system.

There are several subtypes of EEC including K cells, L cells, I cells, N cells, S cells, M cells, D cells, and enterochromaffin cells. K cells are known to promote triglyceride storage including through the secretion of gastric inhibitory peptide. L cells are primarily found in the ileum and large intestine and are known to secrete glucagon-like peptide-1 (GLP-1), pancreatic peptide YY₃₋₃₆, oxyntomodulin, and glucagon-like peptide 2. I cells are primarily located in the duodenum and jejunum, secrete cholecystokinin (CCK), and are known to modulate, inter alia, bile secretion and satiety. N cells are primarily located in the jejunum and secrete neurotensin which is known to modulate smooth muscle cell contraction. S cells are located primarily in the jejunum and duodenum and are known to secrete secretin. M cells are also located primarily in the jejunum and duodenum and are known to secrete motilin. D cells are located primarily in the small intestine and secrete somatostatin. Enterochromaffin cells are known to play a significant role in intestinal motility and are known to secrete serotonin and histamine.

Nervous System Cells

The number of neurons within the enteric nervous system (ENS) rivals that of the neurons found within the spinal cord. Bidirectional interactions between the central nervous system and the gastrointestinal tract (GI) are beginning to surface; the connections that form these interactions at the enteric nervous system surrounding the GI tract are unmapped. An assortment of neurodevelopmental disorders are associated with disturbances in the gastrointestinal system. Current intestinal systems for studying the enteric nervous system rely on heavy use of in vivo murine and embryonic chick systems or in vitro cell culture of both human and murine cells for implantation. The microenvironment, has been shown to provide geometric tunability for cellular cultures, mimicking physiorelevant conditions. Tissue engineered systems offer the potential for a technology that encompasses the properties of the endogenous human system in a modular, tunable in vitro model. Therefore, we disclose the development of an in vitro human innervated intestinal tissue model that encompasses both human induced neural stem cells (hiNSCs) that may be differentiated into pertinent enteric nervous system neural cell types, as well as enterocyte and goblet cells that create the intestinal epithelial layer. This alternative to the current experimental landscape provides technology that may be used to understand neural circuits controlling the intestine.

As discussed herein, innervation is a critical part of intestinal health and function. It is known that the intestine is central to human health and the enteric nervous system functions as the main component of the autonomic nervous system. As such, intestinal neuromodulation impacts many aspects of human health and the improved understanding of this system provided by aspects of the present invention may well have major implications improving human health. Accordingly, in several embodiments, provided compositions further include a plurality of nervous system cells. In some embodiments, nervous system cells comprise at least one of neurons, glia, and neural stem cells. In some embodiments, at least a plurality of the nervous system cells are functional. In some embodiments, substantially all of the nervous system cells are functional (e.g., capable of firing a plurality of action potentials or differentiating into one or more cell types). In some embodiments, at least some of the plurality of nervous system cells are present across portions of a provided composition, for example, with the cell body in one portion and the axon spanning a second portion. In some embodiments, at least some of the plurality of nervous system cells are present in a gel or hydrogel.

In some embodiments, the nervous system cells are human nervous system cells. In some embodiments, the nervous system cells are or comprise afferent nerve cells. In some embodiments, the nervous system cells are or comprise efferent nerve cells. In some embodiments, the nervous system cells comprise glial cells. In some embodiments, at least some of the plurality of nervous system cells provide functional innervation to at least some of the enterocytes, Paneth cells, enteroendocrine cells, and/or Goblet cells.

In some embodiments, the function of at least some of the plurality of nervous system cells may be assessed via any known method. For example, in some embodiments, the function of at least some of the plurality of nervous system cells may be assessed via real time calcium imaging, magnetic resonance imaging, assays of metabolic function including neurotransmitter production, etc.

In some embodiments, at least some of the plurality of nervous system cells may be at least partially myelinated. In some embodiments, at least some of the plurality of nervous system cells may be fully myelinated (e.g., with a pattern of myelination substantially similar to those found in vivo). In some embodiments, the degree and/or quality of myelination may be assessed using any known method (e.g., via CARS laser).

In some embodiments, the nervous system cells comprise neuronal nitric oxide synthase (nNOS)-expressing neurons. Prior to the present invention, the only known way to achieve nNOS expression in an in vitro system was through direct transplant into mice. As such, this represents the first time development of nNOS expressing neurons has been observed in an entirely in vitro system (e.g., an entirely in vitro system including all human cells).

Inflammation and Gut Signaling

The GI tract is responsible for critical functions beyond just digestive roles. The GI tract also plays critical roles in endocrine, immune and barrier functions. Due to the large surface area exposed to intestinal content, the GI tract is a major entry point for pathogen invasion; the system has several levels of defenses, the first of which is the stratified mucus layer which, alongside the epithelial cells, provides physical protection. Past work indicates that a neural driven cholinergic anti-inflammatory pathway directly modulates the systemic response to pathogen invasion. In the GI tract the net response of nerve activation upregulates the mucosal defense, demonstrating its importance in pathogenesis prevention. An assortment of challenges arise in studying the intestine through in vitro models, including maintenance of the ENS and the structure of the microvilli.

ENS neurons innervate the mucosa and gut-associated lymphoid tissue; past studies have indicated that there is a connection between the immune response and ENS presence and function. In inflamed regions of the bowel in patients with inflammatory bowel disease (IBD), there is an increase in enteric neurons. There is a correlation between the severity of intestinal inflammation and the density of the enteric innervation within mouse models. The study of IBD and the links to neuromodulation within the ENS can assist with the development of therapeutics for aliments that involve the pathogenesis of IBD, such as Crohn's disease and ulcerative colitis which can both last for years or be lifelong aliments requiring significant healthcare expenditure throughout the life of the patient.

There is a correlation between the severity of intestinal inflammation and the density of the enteric innervation within mouse models. Prior studies utilize transgenic mice that have greater than normal (NSE-noggin mice, which overexpress noggin under the control of the neuron-specific enolase promoter) or fewer than normal (Hand2(+/−) mice) numbers of neurons in the enteric nervous system. Currently, the mechanisms by which the ENS affects intestinal inflammation are being explored through the intestinal barrier functions, innate immunity, and immunoregulation. Additionally, alterations in the level of mucosal enterochromaffin cell-derived serotonin (5-HT) levels oppose symptoms of intestinal inflammation, which is promoted in the presence of 5-HT. By emulating the conditions found within mouse models in the in vitro tissue model, we can compare the mechanisms present in human cells to the in vivo models.

### Gut-Brain Axis

Previous studies have shown an assortment of correlations between psychological states and gastrointestinal homeostasis. Psychological stress, both acute or chronic, have been shown to affect barrier function resulting in increased permeability which is implicated in both irritable bowel syndrome (IBS) and inflammatory bowel disease (IBD). There is an increased prevalence of IBD in patients with autism spectrum disorders (ASD). In order to maintain homeostasis, the immune system may rely on non-immunological actors such as neuromodulation that provides a link between the peripheral and central nervous systems. The homeostatic role of the ENS is beginning to be further explored with regard to gut-brain bidirectional signaling. As such, bidirectional communication between the nervous system (enteric and at times, central) and GI barrier functions undoubtedly plays a crucial role in gastrointestinal health and function.

Prior to the present invention, there existed in vitro intestinal model systems that utilize, monolayer transwell culture, organotypic slices, gut-on-a-chip technology, and microfluidic designs. In contrast, in some embodiments, the present invention provides developed, fully functional intestinal systems (see Example 3), that can function for months in vitro, generate suitable digestive enzymes and mucous, and supports studies of infectious diseases and microbiome interactions (unpublished data).

TABLE 1
Previous Intestinal Models

| Intestinal Model | Drawbacks | References |
|---|---|---|
| In vitro transwell | Regardless of use of cell line or primary cells, | (16, 17) |
| monolayer intestinal | systems do not provide the extracellular | |
| system | architecture needed to simulate endogenous | |
| | conditions | |
| Organotypic slice model | Slices are utilized over short term experiments, | (8) |
| | there is no differential oxygen exposure, or the | |
| | ability to study the microbiome in conjunction | |
| | with the system | |
| Gut-on-a-chip model | Model does not integrate the enteric nervous | (18) |
| | system to study barrier function | |
| Microfluidic design | Model does not integrate the enteric nervous | (19) |
| | system to study barrier function | |

Enteric neurons and enteric glia are the main cell types housed within the two ganglionated plexuses in the intestinal wall, the myenteric plexus sandwiched between the longitudinal and circular muscles and the submucousal plexus lying in the submucosa. The addition of ENS layers into an in vitro 3D intestine model will allow researchers to broaden the range of preclinical studies that may be performed in the context of gastrointestinal disease treatments, including those studies involving the use of gut microbiome.

Silk & Silk Fibroin

Compositions provided herein include compositions comprising a plurality of different cells types along with at least one silk fibroin scaffold. Silk fibroin, derived from Bombyx mori silkworm cocoons, is a biocompatible and biodegradable material that degrades slowly in the body, is readily modified into a variety of formats, and generates mechanically robust materials.

As used herein, the term “fibroin” includes, but is not limited to, silkworm fibroin and insect or spider silk protein. In some embodiments, fibroin is obtained from a solution containing a dissolved silkworm silk or spider silk. In some embodiments silkworm silk protein is obtained, for example, from Bombyx mori, and spider silk is obtained from Nephila clavipes. In some embodiments, silk proteins suitable for use in the present invention may be obtained from a solution containing a genetically engineered silk, such as from bacteria, yeast, mammalian cells, transgenic animals or transgenic plants. See, for example, WO 97/08315 and U.S. Pat. No. 5,245,012.

In some embodiments, silk fibroin scaffolds comprising silk fibroin may be made using one or more silk solutions, which are known to be highly customizable and allow for the production of any of a variety of end products. As such, in some embodiments, provided compositions may be produced using any of a variety of silk solutions. Preparation of silk fibroin solutions has been described previously, e.g., in WO 2007/016524, which is incorporated herein by reference in its entirety. The reference describes not only the preparation of aqueous silk fibroin solutions, but also such solutions in conjunction with bioactive agents.

In accordance with various embodiments, a silk solution may comprise any of a variety of concentrations of silk fibroin. In some embodiments, a silk solution may comprise 0.1 to 30% by weight silk fibroin. In some embodiments, a silk solution may comprise between about 0.5% and 30% (e.g., 0.5% to 25%, 0.5% to 20%, 0.5% to 15%, 0.5% to 10%, 0.5% to 5%, 0.5% to 1.0%) by weight silk fibroin, inclusive. In some embodiments, a silk solution may comprise at least 0.1% (e.g., at least 0.5%, 1%, 2%, 3%, 4%, 5%, 6%, 7%, 8%, 9%, 10%, 15%, 20%, 25%) by weight silk fibroin. In some embodiments, a silk solution may comprise at most 30% (e.g., at most 25%, 20%, 15%, 14%, 13%, 12%, 11%, 10%, 5%, 4%, 3%, 2%, 1%) by weight silk fibroin.

In accordance with various embodiments, the compositions disclosed herein can comprise any amount/ratio of silk fibroin to the total volume/weight of the overall composition. In some embodiments, the amount of silk fibroin in the solution used for making a provided silk fibroin composition itself can be varied to vary properties of the end silk fibroin composition. By way of specific example, in some embodiments, silk fibroin comprises at least 1% of a provided composition by weight (e.g., at least 2%, 3%, 4%, 5%, 6%, 7%, 8%, 9% 10%, 15%, 20%, 25% or more). In some embodiments, silk fibroin comprises at most 35% of a provided composition by weight (e.g., at most 30%, 25%, 20%, 15%, 10%, 5% or less). In some embodiments, silk fibroin comprises between 1-35% of a provided composition by weight (e.g., between 1-30%, 1-25%, 1-20%, 1-15%, 1-10%, 1-5%, 5-25%, 5-20%, 5-15%, 5-10%). In some embodiments, silk fibroin comprises 4-5% silk fibroin by weight.

Silk fibroin solutions used in methods and compositions described herein may be obtained from a solution containing a dissolved silkworm silk, such as, for example, from Bombyx mori. Alternatively, a silk fibroin solution is obtained from a solution containing a dissolved spider silk, such as, for example, from Nephila clavipes. Silk fibroin solutions can also be obtained from a solution containing a genetically engineered silk. Genetically engineered silk can, for example, comprise a therapeutic agent, e.g., a fusion protein with a cytokine, an enzyme, or any number of hormones or peptide-based drugs, antimicrobials and related substrates.

In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 100° C., 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected, is present in an amount that is below detection, or is absent.

If necessary, the silk solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, provided silk compositions described herein, and methods of making and/or using them may be performed in the absence of any organic solvent. Thus, in some embodiments, provided compositions and methods are particularly amenable to the incorporation of labile molecules, such as bioactive agents or therapeutics, and can, in certain embodiments, be used to produce controlled release biomaterials. In some embodiments, such methods are performed in water only.

In some embodiments, the silk fibroin solution can be produced using organic solvents. Such methods have been described, for example, in Li, M., et al., J. Appl. Poly Sci. 2001, 79, 2192-2199; Min, S., et al. Sen'l Gakkaishi 1997, 54, 85-92; Nazarov, R. et al., Biomacromolecules 2004 5, 718-26, contents of all which are incorporated herein by reference in their entireties. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, e.g., solvents other than water.

In some embodiments, biocompatible polymers can also be added to a silk solution to generate composite materials in the methods and processes of the present invention. Exemplary biocompatible polymers useful in some embodiments of the present invention include, for example, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419). In some embodiments, two or more biocompatible polymers can be used.

Various embodiments may comprise one or more silk fibroin scaffolds which comprise pores of various sizes (i.e., porous silk scaffold). In some embodiments, pores in a three dimensional silk scaffold have a diameter between about 1-1,000 μm, (e.g., between about 1-100, 20-100, 30-100, 40-100, 50-100, 60-100, 70-100, 80-100, 90-100, 50-1,000, 100-1,000, 200-1,000, 300-1,000, 400-1,000, 500-1,000, 600-1,000, 700-1,000, 800-1,000, or 900-1,000 μm) inclusive. In some embodiments, pores in a silk scaffold have a diameter between about 100-1,000 μm, inclusive. In some embodiments, pores in a silk scaffold have a diameter between about 100-300 μm, inclusive. In some embodiments, pores in a silk scaffold have a diameter between about 150-250 μm, inclusive. In some embodiments, pores may be interconnected in particular provided compositions.

In some embodiments, silk scaffolds may be made porous through the use of one or more porogens. It is contemplated that any known porogen may be suitable for use according to various embodiments. In some embodiments, a porogen may be or comprise crystals (e.g., sodium chloride crystals), micro- and/or nano-spheres, polymers (such as polyethylene oxide, or PEO), ice crystals, and/or a laser. In some embodiments a porogen may comprise mechanical introduction of pores (e.g., using a needle or other article or device to pierce a scaffold one or more times, or using stress to introduce one or more tears in a scaffold).

In accordance with various embodiments, provided silk fibroin scaffolds (e.g., porous silk scaffolds) may be of a variety of different thicknesses. In some embodiments, a silk scaffold is less than or equal to 100 cm thick. In some embodiments, a silk scaffold is between 0.1 and 100 cm thick (e.g., 0.2-100, 0.5-10, 0.2-9, 0.2-8, 0.2-7, 0.2-6, 0.2-5, 0.2-4, 0.2-3, 0.2-2, 0.2-1, 0.5-1, 0.2-0.9, 0.2-0.8, 0.2-0.7, 0.2-0.6, 0.2-0.5, 0.2-0.4, 0.2-0.3 cm thick). In some embodiments, a silk scaffold is about 0.2-0.5 μm thick, inclusive. In some embodiments, a silk scaffold is of a substantially uniform thickness. In some embodiments, a silk scaffold varies in thickness across a particular length (e.g., a 1 cm).

In some embodiments, one or more silk scaffolds (and/or silk solutions from which a scaffold is made) may comprise, for example, low molecular weight silk fibroin fragments (e.g., fragments of silk fibroin between 3 kDa and 200 kDa), though any molecular weight silk may be used in accordance with various embodiments. In any of the embodiments described herein, silk fibroin fragments can include one or more mutations and/or modifications, relative to a naturally occurring (e.g., wild type) sequence of silk fibroin. Such mutation and/or modification in the silk fibroin fragment can be spontaneously occurring or introduced by design. For example, in some embodiments, such mutation and/or modification in the silk fibroin fragment can be introduced using recombinant techniques, chemical modifications, etc.

Silk—Conformational Changes

In some embodiments, a conformational change can be induced in the silk fibroin to control the solubility of the silk fibroin composition. In some embodiments, the conformational change can induce the silk fibroin to become at least partially insoluble. Without wishing to be bound by a theory, an induced conformational change may alter the crystallinity of the silk fibroin, e.g., Silk II beta-sheet crystallinity. In accordance with various embodiments, the conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al; Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Contents of all of the references listed above are incorporated herein by reference in their entireties.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low beta-sheet content using conditions at 4° C. (a helix dominated silk I structure), to higher beta-sheet content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Water or water vapor annealing is described, for example, in PCT application no. PCT/US2004/011199, filed Apr. 12, 2004 and no. PCT/US2005/020844, filed Jun. 13, 2005; and Jin et al., Adv. Funct. Mats. 2005, 15: 1241 and Hu et al., Biomacromolecules, 2011, 12(5): 1686-1696, contents of all of which are incorporated herein by reference in their entireties

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with shear stress. The shear stress can be applied, for example, by passing the silk composition through a needle. Other methods of inducing conformational changes include applying an electric field, applying pressure, or changing the salt concentration.

In some embodiments, alteration in the conformation of the silk fibroin can be induced by horseradish peroxidase (HRP) and hydrogen peroxide (H₂O₂). As is known in the art, HRP facilitates crosslinking of the tyrosines in silk fibroin via the formation of free radical species in the presence of hydrogen peroxide. Exemplary methods may be found in Partlow et al., Highly tunable elastomeric silk biomaterials, 2014, Adv Funct Mater, 24(29): 4615-4624.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin may comprise a protein structure that substantially includes β-turn and β-strand regions. Without wishing to be bound by a theory, the silk β sheet content can impact gel function and in vivo longevity of the composition. It is to be understood that composition including non-β sheet content (e.g., e-gels) can also be utilized. In some embodiments, the silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% (3-turn and β-strand regions, or at least 95% β-turn and β-strand regions. In yet other aspects of these embodiments, the silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk β sheet content, from less than 10% to ˜55% can be used in the silk fibroin compositions disclosed herein.

In some embodiments, the silk fibroin has a protein structure that is substantially-free of α-helix and random coil regions. In aspects of these embodiments, the silk fibroin has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

Antimicrobial Responses

In accordance with various embodiments, some provided compositions are capable of initiating an antimicrobial defense. In some embodiments, initiation of an antimicrobial defense may be or comprise expression of one or more genes and/or proteins that are associated with a host's response to a microbial insult. Human intestines are constantly exposed to a vast number and diversity of bacteria. To cope with the substantial microbial threats, the intestinal epithelium uses defense mechanisms which involve the activation of a number of microbial recognition and innate immune pathways, the secretion of diverse proinflammatory cytokines/chemokines and antimicrobial proteins to kill or prevent the growth of bacteria in infected tissues. As shown in the Examples below, certain provided compositions can exhibit significant antimicrobial (e.g., antibacterial) responses, as evidenced by the increased expression of genes with important roles in pathogen recognition and the activation of immune responses, including microbial sensor genes, cytokines, inflammatory mediator genes, downstream signal transduction genes, and inflammasome signaling genes.

In some embodiments, an antimicrobial defense is or comprises upregulated gene and/or protein expression of one or more of lymphocyte antigen 96 (LY96), toll-like receptor-2 (TLR2), toll-like receptor-4 (TLR4), toll-like receptor-5 (TLR5), toll-like receptor-6 (TLR6), c-reactive protein (CRP), deleted in malignant brain tumors-1 (DMBT1), interferon regulatory factor-7 (IRF7), z-DNA-binding protein 1 (ZBP1), chemokine (C-C motif) ligand 3 (CCL3), C-X-C motif chemokine 1 (CXCL1), C-X-C motif chemokine 2 (CXCL2), interleukin-12 subunit alpha (IL12A), interleukin-12 subunit beta (IL12B), interleukin 1 beta (IL1B), interleukin 6 (IL6), myeloid differentiation primary response gene 88 (MYD88), nucleotide-binding oligomerization domain-containing protein 1 (NOD1), nucleotide-binding oligomerization domain-containing protein 2 (NOD2), Ras-related C3 botulinum toxin substrate 1 (RAC1), p65 (RELA), tumor necrosis factor (TNF), bactericidal permeability-increasing protein (BPI), cathelicidin (CAMP), cathepsin G (CTSG), lysozyme (LYZ), myeloperoxidase (MPO), secretory leukocyte protease inhibitor (SLPI), mitogen-activated protein kinase kinase 1 (MAP2K1), mitogen-activated protein kinase 1 (MAPK1), mitogen-activated protein kinase 8 (MAPK8), JUN, killer cell immunoglobulin-like receptor subunit a (NKB1A), caspace 1 (CASP1), and apoptosis-associated speck-like protein containing a CARD (PYCARD).

Integration with Devices

In some embodiments, provided methods and compositions include one or more electrical devices, optical devices, and/or optical tools. In some embodiments, provided compositions may comprise an electrical device that is functionally connected to at least some of a plurality of nervous system cells. In some embodiments, an electrical device, optical device, or optical tool comprises silk fibroin. By way of specific, non-limiting examples, in some embodiments, provided compositions may comprise one or more of an electrode, a multi-electrode array (e.g., an interdigitated array), a sensor, and an air pump.

In some embodiments, integrated multi-electrode arrays may be used with the intestinal equivalents in vitro to conduct and map signal collection over time, conduct signal processing and modeling related to normal vs. abnormal (e.g., during inflammation) states, applying signaling regimes to modulate intestine functions and outcomes. Incorporation of multi-electrode arrays allows tracking of neuronal signaling in response to normal vs abnormal intestinal functions with models used to alter normal intestinal outcomes via application of appropriate neural activation patterns. Multi-electrode arrays in vivo may also be used to conduct wireless assessments of signaling regimes into murine model of inflammation in hyperplastic and wild type ENS. Alterations in conditions in murine models may be compared to the in vitro innervated intestinal model, and assessments made to determine how application of the signaling regimes can change intestinal functions.

The ability to monitor the state of the tissue with conformal silk electrode arrays (surgically implanted or wrapped around tissues) allows for the development of quantitative, mathematical/computational models for mapping as a function of nerve activity in space and time for the state of the tissue. While this processing problem may be cast as one of classification (normal, wound, inflammation), it may well be that subtler variability can be reliably characterized (e.g., severity of a wound or inflammation) in which case regression methods are more appropriate. In either case, the processing approach is the same: reduction of the raw data in a manner that is adapted to the identification of specific information and the development of classification and/or regression techniques for extracting the specific information of interest. Building on our previous efforts constructing such models and processing methods in the context of subsurface sensing, our work here will focus on the development of low dimensional manifold techniques for data reduction coupled with appropriate methods for manifold-based classification and/or regression. Initial efforts will focus on determining the information content of the data to quantify the achievable resolution; i.e., our ability to reliably characterize the state of the tissue at two points in space and time after which we will concentrate on the development of real-time processing methods that can be used in the context of closed-loop stimulation and control experiments.

Methods of Making

In some embodiments, the present invention provides methods including the steps of providing a silk fibroin scaffold, associating a plurality of fibroblasts with the silk fibroin scaffold, associating a plurality of intestinal stem cells with the silk fibroin scaffold, differentiating the plurality of intestinal stem cells into two or more of enterocytes, Goblet cells, Paneth cells, and enteroendocrine cells to form an intestine-like composition. In some embodiments, intestinal stem cells do not include totipotent stem cells. In some embodiments, intestinal stem cells do not include pluripotent stem cells. In some embodiments, intestinal stem cells do include multipotent stem cells (i.e., cells capable of differentiating into enterocytes, Goblet cells, Paneth cells, and/or enteroendocrine cells). In some embodiments, the intestinal stem cells are differentiated into three or more of enterocytes, Goblet cells, Paneth cells, and enteroendocrine cells to form an intestine-like composition.

In some embodiments, provided methods further include a step of associating a plurality of nervous system cells with the silk fibroin scaffold. In some embodiments, provided methods further comprise differentiating at least some of the plurality of nervous system cells into one or more of afferent nerve cells and efferent nerve cells. In some embodiments, the nervous system cells are human nervous system cells. In addition to the above, provided methods encompass all of the various parameters and disclosure herein with respect to provided compositions.

Methods of Using

In some embodiments, provided methods and compositions provide physiologically relevant systems for study and characterization of diseases, disorders, or conditions of the enteric system. For example, in some embodiments, provided methods and compositions may be useful in studying, inter alia, intestinal cancers, nutrition, bacterial additions for microbiome impacts, impact of drugs on intestinal functions (e.g., inflammatory cells to emulate inflammatory bowel disease), and therapeutic aspects thereof, including discovery and/or characterization of new therapeutic strategies.

In some embodiments, provided methods include the steps of providing a composition in accordance with those described herein (e.g., a composition comprising a plurality of enterocytes, a plurality of fibroblasts, a plurality of Goblet cells, a plurality of Paneth cells, a plurality of enteroendocrine cells, and a silk fibroin scaffold), exposing the composition to one or more therapeutic agents (e.g., in or through a lumen therein), and characterizing the response of one or more of the enterocytes, Goblet cells, Paneth cells, and enteroendocrine cells to the one or more therapeutic agents. In some embodiments, a therapeutic agent that may be useful in various applications may be or comprise one or more of anti-inflammatory drugs (e.g., for treating inflammatory bowel disease), anti-cancer agents, probiotic compounds, nutraceutical compounds, aminosalicylates, corticosteroids, antimicrobial compounds (e.g., antibacterial, antiviral, and/or antifungal compounds). In some embodiments, a therapeutic agent may be any drug or other therapeutic being considered or oral delivery (i.e., to test the effects thereof on intestinal tissue).

Particularly when a provided composition is being used as a disease modal, in some embodiments, at least some of the enterocytes, Goblet cells, Paneth cells, and/or enteroendocrine cells of a provided composition exhibit one or more pathologic abnormalities as compared to similar cells from a healthy individual. In some embodiments, the one or more pathologic abnormalities is indicative of or correlated to the presence of a disease. In some embodiments, the disease is inflammatory bowel syndrome, Celiac Disease, Crohn's disease, intestinal cancer, intestinal ulcer, ulcerative colitis, and diverticulitis.

### EXAMPLES

### Example 1—Exemplary Methods and Compositions

Bioreactor Design and Fabrication

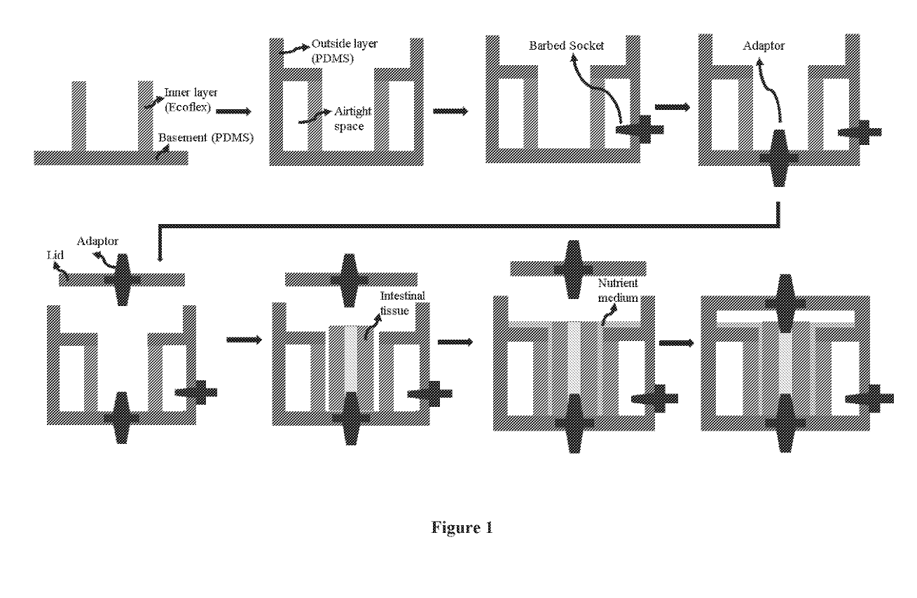

In this Example, the implementation of certain embodiments of provided methods and compositions is described. Specifically, in this Example, Teflon made rods and tubes were used to construct a mold first. The basement layer was prepared by casting PDMS prepolymer (10:1 w/w ratio of PDMS to curing agent) in the mold and the inner layer walls were prepared by casting ECOFLEX® 00-30 silicone rubber (1:1 w/w ratio of part 1A and part 1B). The ECOFLEX® was highly stretchable rubber and PDMS was less deformable. After the inner layer wall formed, PDMS was used again to form the outside layer of the bioreactor. An airtight chamber was created between the inner layer wall and outside layer wall. A barbed socket was mounted to the outside layer. In the center of the basement, an adaptor was mounted on the bottom layer of the scaffold. The upper end of this adaptor inside the inner layer was used to fit into the lumen of the tissue scaffold; the lower end of this adaptor was used to connect the tube which reserve the waste medium. A lid was used to cover the bioreactor. In the center of the lid, another adaptor was mounted in the center of the lid. The lower end of this adaptor inside the bioreactor was used to fit the lumen of the scaffold, and the other end of this adaptor was used to connect the tube to transport the medium. Before close of the lid, the lumen of the intestinal tissue was set into the upper end of the adapter in the basement of the bioreactor and nutrient medium was filled in the rest space. When the lid in the bioreactor was closed, the lower end of the adaptor in the lid would also fit into the lumen of the scaffold (see FIG. 1).

Function of the Bioreactor

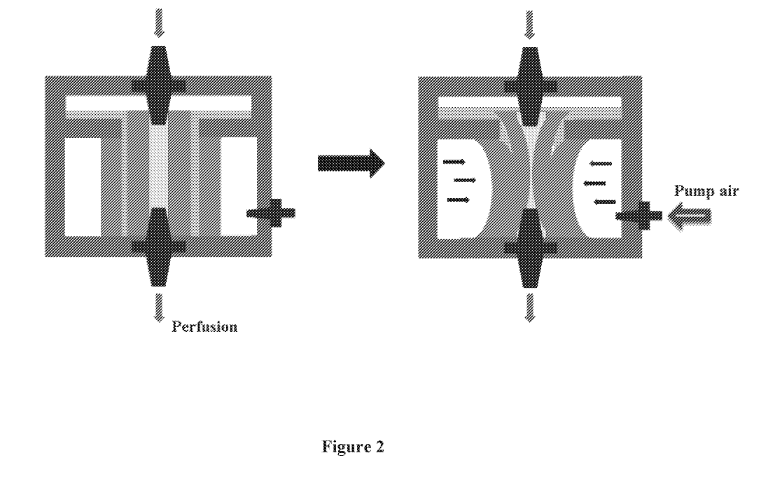

Among the advantages provided by some embodiments of the present invention, the bioreactor exemplified in this Example highlights at least two new and powerful physiologically relevant features, a perfusion system and peristaltic motion system. For the exemplary perfusion system described in this Example, the intestinal tissue was fit between two adaptors. The epithelium culture medium was circulated at a constant flow rate of 30 μL/h, which corresponds to a shear stress of 0.02 dyne/cm², was perfused through the lumen of the scaffold. This flow was sufficient to provide nutrient support to the epithelial layer of the tissue. The rest space in the bioreactor was filled with nutrient medium to support the myofibroblast in the sponge of the tissue. After the first two weeks, until the epithelium was well formed, the perfusion medium was changed to simulated intestinal fluid, which better mimics the physiology of the systems. For the exemplary peristaltic motion system described in this Example, a tube was used to connect the socket and the syringe pump which pump and plug air into the chamber to create a different air pressure inside the chamber and outside environment. Therefore, the highly stretchable ECOFLEX® could provide mechanical stimulation to the tissue inside the inner wall. The frequency of the deformation was set to 2 cycle/min, 5 cycle/min and 10 cycle/min separately (see FIG. 2).

Integrating Oxygen Control System to Bioreactor

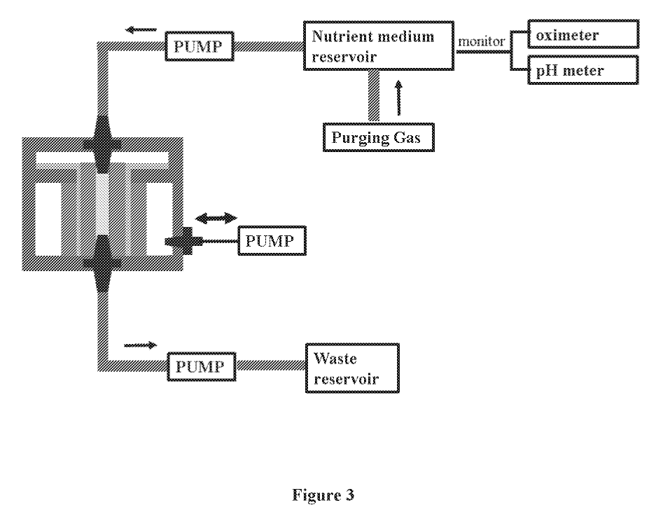

In the oxygen control system described in this Example, a media container, serving as a perfusion liquid reservoir, was sealed and purged with a gas mixture with the desired oxygen level for 24 hours prior to perfusion. To create a microaerophilic lumen environment, a gas mixture of 5% CO₂, <5% O₂, >90% N₂ was used to pre-equilibrate the luminal media. To create an anaerobic lumen environment, the luminal perfusion medium was equilibrated with <1% O₂, 5% CO₂, 90% N₂ gas. To create a fully anaerobic environment in the lumen (<0.1%), the oxygen was reduced to the lowest level we could attain by perfusion. The luminal perfusion medium was equilibrated with 4% H₂, 5% CO₂, 91% N₂ gas. The oxygen levels in each perfusion medium were monitored with a Microx TX3 oximeter (PreSens, Regensburg, Germany). The purge gas compositions may be tailored to meet specific needs in engineering of a particular desired luminal-epithelial oxygenation profile. The pHs of the perfusion media were checked using a SevenMulti benchtop pH meter (Mettler-Toledo; Columbus, Ohio, USA) immediately before and after purging. Medium was collected in waste bottles after perfusion for further analysis (FIG. 3).

Tissue Culture and Incubation

The tissue culture procedure was a modified version of certain previously used methods (see Chen et al., Robust bioengineered 3D functional human intestinal epithelium, Scientific Reports, 2015, 5, the disclosure of which is hereby incorporated in its entirety). Briefly, the hollow channel of the 3D scaffolds was used to accommodate human intestinal epithelial cells (Caco-2/HT29-MTX cells (3:1)), while the porous bulk space was used, inter alia, to house primary human intestinal myofibroblasts (H-InMyoFibs). After implanting the cells in the scaffold, the tissues were housed in the well plate with a nutrient medium (DMEM: SMGM (1:1)) for 24 hours. Subsequently, the tissue was set into the bioreactor. For the short term study (2 weeks), the epithelium culture medium was perfused at a constant flow rate of 30 μL/h through the lumen of the scaffold. The frequency of air pumping was set to either 2 cycle/min, 5 cycle/min or 10 cycle/min separately. For the long term study, the epithelium culture medium was perfused at a constant flow rate of 30 μL/h through the lumen of the scaffold in the first 2 weeks. After that, the perfused medium was changed to simulated intestinal fluid with the same perfusion speed. The frequency of the air pumping was set to 2 cycle/min to compare control sample (static, perfuse only, without peristaltic associated movement).

Results:

Short Term (2 Weeks)

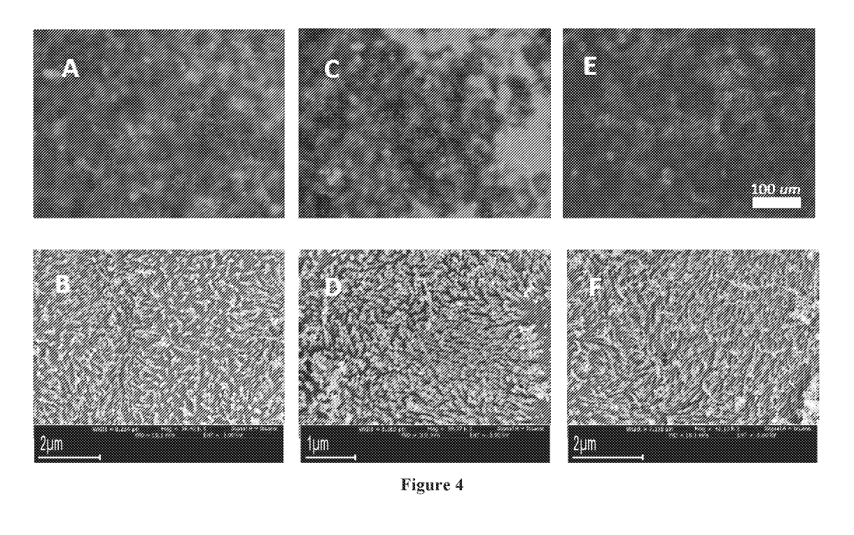

At 2 weeks' time point,
Tight junction were identified in all three groups (FIG. 4, panel A: 0 cycle/min, 4C: 2 cycle/min, 4E: 5 cycle/min)
The number of villus in mechanical stimulation group (FIG. 4, panel D: 2 cycle/min and FIG. 4, panel F: 2 cycle/min) were higher than control group (FIG. 4, panel B: 0 cycle/min)

Long Term (5 Weeks)

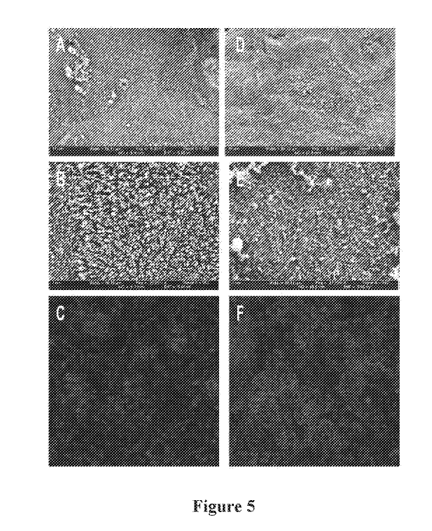

At 5 weeks incubation,
The microvillus brush border on both control group and mechanical stimulation group were still identified, indicating the well-formed tight epithelium layer. (FIG. 5, panels A and D)
The tight junction on two control groups were also identified. (FIG. 5, panels C and F)
Villus in mechanical stimulation group are more polarized than the group. (FIG. 5, panels B and E)

As shown above, in this Example, provided compositions are capable of forming tight junctions and villi, and also of reacting to mechanical stimulation of a peristaltic nature.

### Example 2—Development of Functional Intestinal Cell Subpopulations

In this Example, it is described that certain provided compositions are able to support differentiation of seeded cells into at least four major subpopulations of the intestinal epithelium as well as formation of a secreted mucus layer.

Methods:

Culture of Human Primary Large and Small Intestinal Epithelial Cells:

Human small intestine primary cells were purchased from ScienCell (CAT#2950), while the human large intestine primary cells were purchased from CellBiologics (CAT# H-6051). Both cells were cultured in medium that came with the cells from the manufacturer. Medium was changed every other day. Cells were used at passage 3. Both of small and large intestinal epithelial cells were seeded on 3D scaffold lumen at a density of 2×10⁶ cells/mL and cultured up to 2 weeks.

Enteroid/Colonoid Expansion, Primary Cell Isolation, Monolayer Formation and Differentiation:

Enteroids and colonoids were maintained in Matrigel in conditioned medium containing Wnt 3, Rspo-1, and Noggin conditioned media, B27 supplement, N2 supplement, n-acetylcysteine, EGF, Gastrin, Nicotinamide, A83, and SB202190). Cells were passaged approximately every 6 to 7 days at a 1:5 ratio. To form enteroids/colonoids monolayers, human enteroids/colonoid (˜1-2 wells of a 24-well plate per Transwell) were dissociated using trypsin and strained through 40 μm cell strainers. Primary cells were subsequently isolated from both human intestinal enteroids and colonoids and seeded on the luminal surfaces of our 3D scaffold systems. The systems were maintained in conditioned medium (containing Wnt 3, Rspo-1, and Noggin conditioned media, B27 supplement, N2 supplement, n-acetylcysteine, EGF, Gastrin, Nicotinamide, A83, SB202190, 10 uM Y-27632) overnight and differentiation medium (conditioned medium without Wnt 3a, Nicotinamide and SB202190, 50% of R-Spondin and Noggin conditioned medium) for 5 days.

Results:

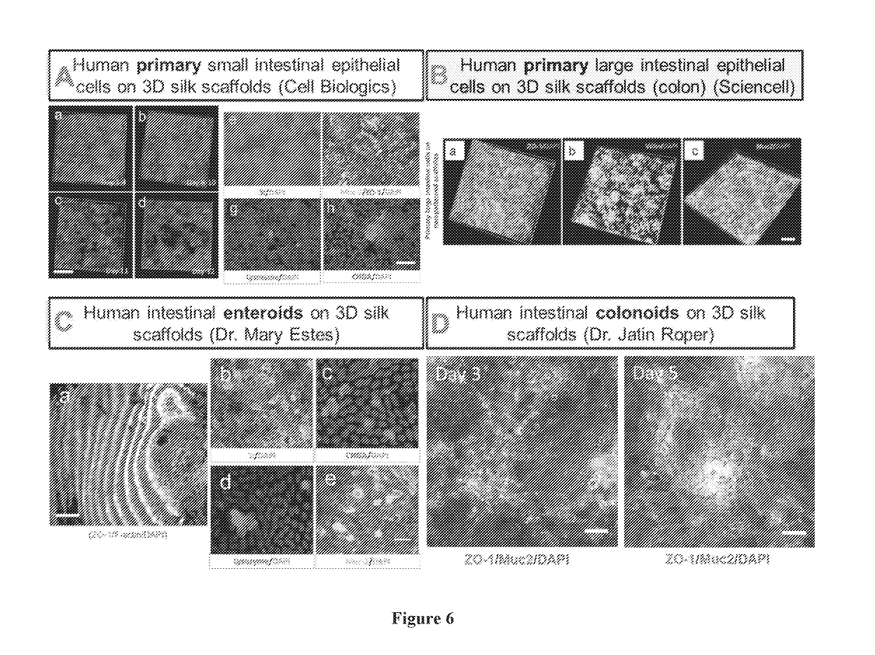

Cultivation of Human Primary Large and Small Intestinal Epithelial Cells in 3D Silk Scaffolds (FIG. 6, panels A and B)—

During co-culture of the small primary cells with human intestinal myofibroblasts, tissue constructs were maintained up to 10 days (FIG. 6, panel A [a-d]). The primary small intestinal epithelium grown on the hollow channels was assayed for expression of sucrase-isomaltase (SI) (FIG. 6, panel A [e]), ZO-1, Mucin 2 (FIG. 6, panel A [f]), lysozyme (FIG. 6, panel A [g]) and chromogranin A (CHGA) (FIG. 6, panel A [h]) to identify four major subpopulations of intestinal epithelium; Enterocytes, Goblet cells, Paneth cells, and enteroendocrine cells. All of these markers were detected on the primary small intestinal epithelium system at day 7 post cell seeding. During co-culture of the large primary cells with human intestinal myofibroblasts, tissue constructs were maintained up to around 7 days (FIG. 6, panel B [a-c]). The expanded large intestinal cells formed an integrate monolayer on the lumen and expressed serval intestinal biomarkers, such as ZO-1, villin and Mucin 2.

Cultivation of Human Intestinal Enteroids and Colonoids in the 3D Silk Scaffolds (FIG. 6, panels C and D)—

We used primary cells generated by enteroids and colonoids isolated from human intestinal tissues, which can be converted to monolayer cultures and then used in our 3D scaffolds to generate the intestinal tissues. After cell seeding, these enteroid/colonoid-derived scaffolds were tested the expression of intestinal biomarkers by immunostaining and confocal microscopy. The small intestinal epithelial cells isolated from enteroids formed confluent monolayers featuring a typical chicken-wire pattern of ZO-1 expression (FIG. 6, panel C[a]) and covered by a secreted mucus layer (FIG. 6, panel C[e]) when grown on the luminal surface of 3D silk scaffolds. Most importantly, like primary cells, these cells comprise four major subpopulations of intestinal epithelium (enterocytes ((FIG. 6, panel C[b]), Goblet cells (FIG. 6, panel C[e]), Paneth cells (FIG. 6, panel C[d]), and enteroendocrine cells (FIG. 6, panel C[c])) after being cultured in the differentiation medium. The large intestinal primary epithelial cells isolated from colonoids also attached to the 3D scaffolds and formed confluent epithelial monolayers. After culturing in differentiation medium for 3 days (FIG. 6, panel D [left]), the monolayer expressed the tight junction protein ZO-1, and a major component of mucus Muc-2. At day 5 after differentiation (FIG. 6, panel D [right]), a higher level of Muc-2 expression was observed in the epithelium compared to day 3.

### Example 3—Further Exemplary Compositions and Methods Including Electronic Devices

In this Example, certain exemplary embodiments are described which include at least one electronic device.

Engineered Intestinal Tissue Model—

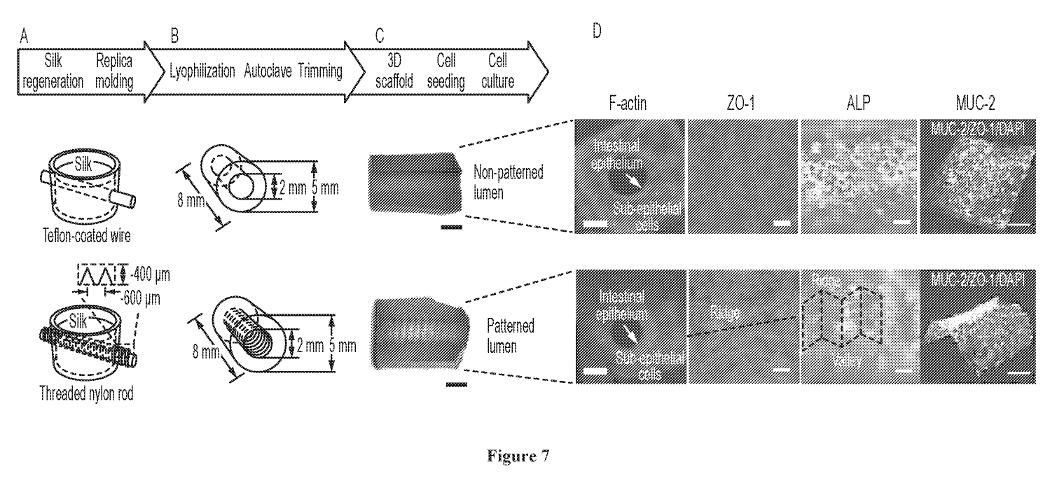

In this Example, a robust bioengineered 3D functional human intestinal epithelium is described that allows for the accumulation of mucous secretions on the epithelial surface, establishes a low oxygen tension in the lumen, and allows for a surface to culture gut-colonizing bacteria. An outline of the system is indicated in FIG. 7, panels A-D. The addition of ENS layers into our in vitro 3D intestine model will allow us to broaden the range of preclinical studies that may be performed in the context of neural circuit elucidation and gastrointestinal disease treatments.

Human Induced Neural Stem Cells (hiNSCs)—

We have generated stable human induced neural stem cells (hiNSCs) via direct reprogramming of primary fibroblast and adipose-derived stem cells. These hiNSC lines differentiate into Tuj 1, MAP2, and NeuN-positive neurons within 4 days, and mature hiNSCs express synaptic markers as well as generate action potentials. As these cells demonstrate the ability to migrate, engraft, and contribute to the formation of peripheral nervous systems while maintaining the ability to be passaged indefinitely and cryopreserved as colonies, they will be utilized for innervating the intestinal model.

Electronic Interfaces & Conformal Silk Electrodes—

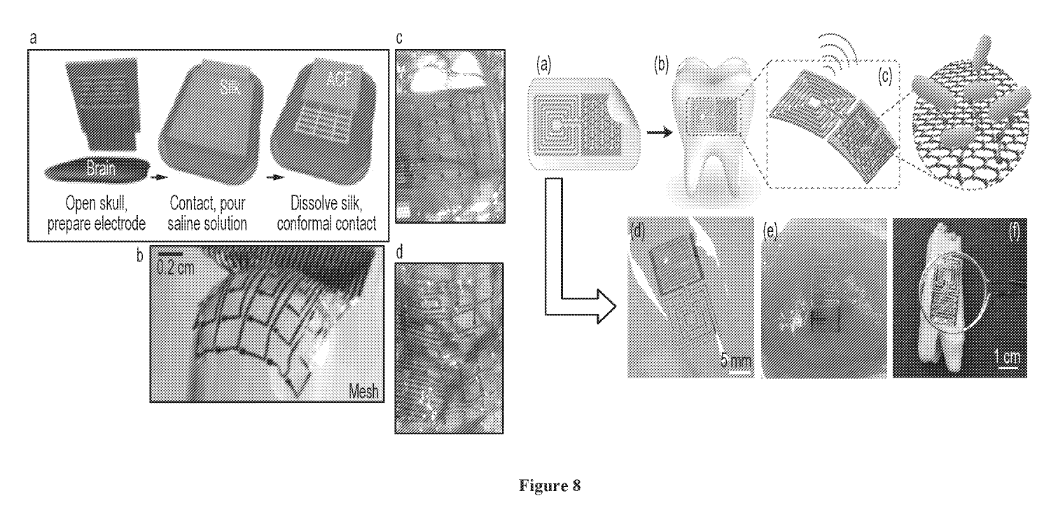

This example demonstrates the ability to interface functional electronic components with living tissue (e.g., intestinal tissue, teeth). Electrodes capable of non-invasive integration with the soft, curvilinear surfaces of biological tissues were demonstrated by devising ultrathin electronic electrode arrays supported by bioresorbable substrates of silk. These devices were placed on the surface of a brain in a feline animal model where spontaneous, conformal wrapping process driven by capillary forces at biotic/abiotic interface resulted in tight interfaces and improved brain signal recordings. The devices also conformally adhered on tooth enamel and allowed for in vivo neural mapping or wireless detection of analytes and stimulation (FIG. 8, panels a-d and (a)-(f)). It is expected that such addition of an electronic interface is equally applicable to compositions provided herein.

### Example 4—Exemplary Compositions Provided, at Least in Part, from Patient Cells

In this Example, unless otherwise specified, the materials and methods used were as follows:

Materials & Methods

Cell Culture:

Human Intestinal Enteroid Culture—

HIEs isolated from human jejunum were kindly provided by Dr. Mary Estes from The Baylor College of Medicine through the Texas Medical Center Digestive Diseases Center Study Design and Clinical Research Core. The Baylor College of Medicine Institutional Review Board approved the study protocol (protocol numbers H-13793 and H-31910). Procedures for maintaining and passaging HIEs were previously described. Briefly, frozen vials containing HIEs were thawed out and resuspended in Matrigel (25 μL/well, Corning). The Matrigel mixture was plated as droplets into 24-well tissue culture plates and incubated at 37° C. for 5-10 minutes to polymerize the Matrigel. 500 μL of HIE growth medium, consisting of 15% Advanced DMEM/F12 (Invitrogen) supplemented with 100 U/ml penicillin-streptomycin (Invitrogen), 10 mM HEPES buffer (Invitrogen), and 1×GlutaMAX (Invitrogen); 10% Noggin-conditioned medium (made from Noggin-producing cells; kindly provided by G. R. van den Brink, Amsterdam, The Netherlands); 20% R-spondin-conditioned medium (R-spondin-producing cells; kindly provided by Calvin Kuo, Palo Alto, Calif.); 50% Wnt3A-conditioned medium produced from ATCC CRL-2647 cells (ATCC); 50 ng/ml epidermal growth factor (EGF) (Invitrogen), 10 mM nicotinamide (Sigma-Aldrich), 10 nM gastrin I (Sigma-Aldrich), 500 nM A-83-01 (Tocris Bioscience), 10 μM SB202190 (Sigma-Aldrich), 1×B27 supplement (Invitrogen), 1×N2 supplement (Invitrogen), and 1 mM N-acetylcysteine (Sigma-Aldrich), was added to each well. HIEs were used at passages 10-40. Live enteroids were imaged with a phase microscope (Leica). Human Intestinal Myofibroblast cell culture—H-InMyoFibs were purchased from Lonza and cultured in SMGM™-2 BulletKit™ medium (Lonza) according to the manufacturer's instructions. Cells were used at the passages of 3-5.

Intestinal Epithelial Cell Line Culture—

The Caco-2 (CRL-2102) cell line was obtained from ATCC, and HT29-MTX cell line was obtained from the Public Health England Culture Collections (Salisbury, Great Britain). Both Caco-2 and HT29-MTX cells were grown in DMEM supplemented with 10% fetal bovine serum, 10 μg/mL human transferrin (Invitrogen), and 1% antibiotics and antimycotics (Invitrogen). For Caco-2 and HT29-MTX, cells from passage number 33-44 were used for the experiments. Primary human intestinal epithelial cell culture—hInEpiCs were purchased from Cell Biologics (Chicago, Ill.) and cultured in complete epithelial cell medium (Cell Biologics) following the manufacturer's protocol. Cell were used at passage 2. All cells were cultured in 37° C., 5% CO₂ humidified atmosphere. The medium was changed every other day.

Generation of 3D Silk Scaffolds:

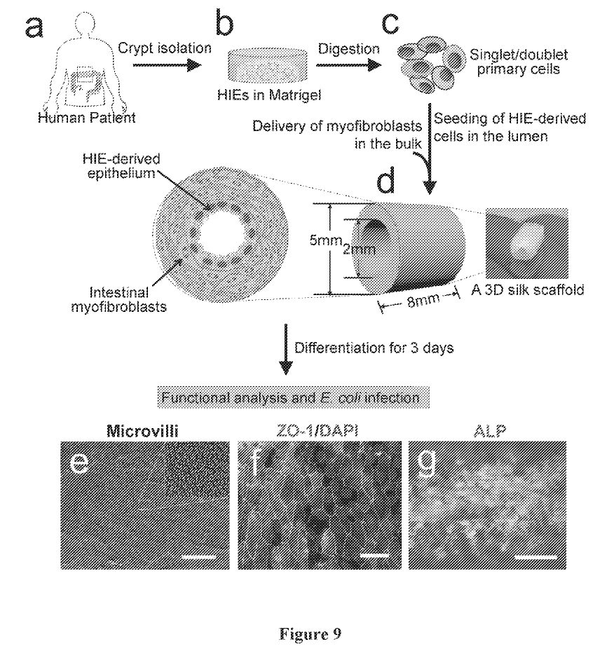

3D silk scaffolds were prepared as described previously. Briefly, silk fibroin was extracted from Bombyx mori silkworm cocoons. To prepare silk scaffolds with hollow channels, special cylindrical molds were cast from polydimethylsiloxane (PDMS; Down Corning). PDMS was prepared by mixing the base reagent with the curing reagent in a mass ratio of 10:1. The cylindrical PDMS molds consisted of a Teflon-coated stainless steel wire (diameter, 2 mm; McMaster-Carr) inserted through the cross section of the cylinder to develop a hollow channel in the silk scaffold. Finally, a 4 to 5% (wt/vol) viscous silk solution was poured into the PDMS molds. The molds were frozen at −20° C. overnight and then transferred to a lyophilizer for drying. The dried silk scaffolds were then autoclaved to induce the β-sheet conformations (insolubility in water), soaked in distilled water overnight, and trimmed along the axis of the hollow channel into a cuboid 5 by 5 by 8 mm. The fabrication method resulted in a scaffold consisting of a hollow channel space (diameter, 2 mm) and a bulk space around the channel that contained interconnected pores (FIG. 9d).

Cell Seeding on 3D Silk Scaffolds:

The general procedures for the seeding of intestinal epithelial cells and myofibroblast cells on 3D silk scaffolds were previously described. The cell seeding strategy for HIE-derived constructs is summarized in FIG. 9. For each HIE-derived scaffold, before being seeded on the scaffolds, 4 wells of HIEs were collected by washing with 500 uL/well of 0.5 mM EDTA (0.5M EDTA diluted 1:1000 in PBS), spun at 1200 rpm at 4° C. for 5 mins. HIEs were then digested with 0.25% Trypsin for 4 mins at 37° C. to obtain pellets containing singlets and doublets for luminal cell seeding. Pellets were resuspended in 354, enteroid growth medium containing 10 μM Y-27632 (Sigma-Aldrich), and then seeded onto each side of the scaffold lumen. Collagen gels containing 2×10⁵ H-InMyoFibs per ml were then delivered into the spongy silk scaffold bulks. HIE-derived scaffolds were first cultured in enteroid growth medium containing 10 μM Y-27632 over night, and then switched to differentiation medium (growth medium without the addition of Wnt 3a, Nicotinamide and SB202190, and with 50% reductions in the concentrations of R-Spondin and Noggin conditioned medium). HIE-derived tissues were cultured in differentiation medium up to 14 days. For cell line-derived and primary cell-derived scaffolds, previously described procedures for the cell seeding in each compartment of the scaffold were exactly followed.

Immunofluorescence and Confocal Imaging:

3D scaffolds with intestinal cells were fixed with 4% paraformaldehyde (PFA, Santa Cruz). Silk scaffolds were cut in half along the longitudinal axis to better expose the lumen to the blocking solutions and antibodies during the following incubation steps. All specimens were then permeabilized using 0.1% Triton X-100 in phosphate-buffered saline (PBS, Invitrogen), then blocked with 5% bovine serum albumin (BSA, Sigma-Aldrich) for 2 hours. These specimens were incubated overnight at 4° C. with anti-human ZO-1(1:100, BD Transduction Laboratories), anti-human-SI (sucrase-isomaltase) (1:100, Santa Cruz Biotech), anti-human-Muc-2 (1:50, Santa Cruz), anti-human-Lyz (1:100, Lysozyme, Abcam) and anti-CghA (1:100, Chromogranin A, Abcam, 1:100), then immersed in Alexa Fluor 488 donkey anti-mouse and Alexa Fluor 546 goat-anti-rabbit secondary antibodies (Invitrogen) at a dilution of 1:250, respectively. Scaffolds were then counterstained with dihydrochloride (DAPI, Invitrogen) before being mounted using Vectashield mounting medium (Vector Laboratories). For live staining, calcein-AM (Invitrogen) was used at different time points, following manufacturer's guidelines. These 3D scaffolds were scanned using a Leica SP2 confocal microscope (Leica Microsystems) and Nikon A1R (Nikon Instruments Inc.) with Z-series capability. Scaffolds were observed under a confocal microscope with a filter set for DAPI (Ex/Em: 350/470 nm), Texas Red (Ex/Em: 540/605 nm) and GFP/FITC (Ex/Em: 488/514 nm). Confocal maximum projection images were assembled with Leica confocal software (ver 2.61, Leica), NIS-Elements AR software package (ver 4.20.01, Nikon) and ImageJ.

Alkaline Phosphatase (ALP) Stain:

ALP staining was performed using the Vector Red Alkaline Phosphatase Substrate Kit I (Vector Laboratories) according to the manufacturer's protocol. Briefly, transwells and silk scaffolds with cells were fixed with 4% PFA for 1 minute at room temperature, then washed two times with PBS. The specimens were incubated with substrate solution at room temperature until suitable staining developed, and were then imaged with the Olympus MVX10 macroscope and captured by CellSens Dimension (ver 1.8.1) program.

Scanning Electron Microscopy (SEM) and Quantification of Microvilli:

Silk scaffolds with cells were cross-linked with 2.5% glutaraldehyde (GA), followed by progressive dehydration in a graded series of ethanol (30%, 50%, 75%, 95% and twice in 100%, 30 minutes at each concentration). The samples were subsequently dried by critical point drying with a liquid CO2 dryer (AutoSamdri-815, Tousimis Research Corp.). Prior to imaging using a scanning electron microscope (Zeiss UltraPlus SEM or Zeiss Supra 55 VP SEM, Carl Zeiss SMT Inc.) at a voltage of 2-3 kV, the samples were coated with a thin layer (10 nm thick) of Pt/Pd using a sputter coater (208HR, Cressington Scientific Instruments Inc.).

Measurement of Oxygen Profiles In Vitro:

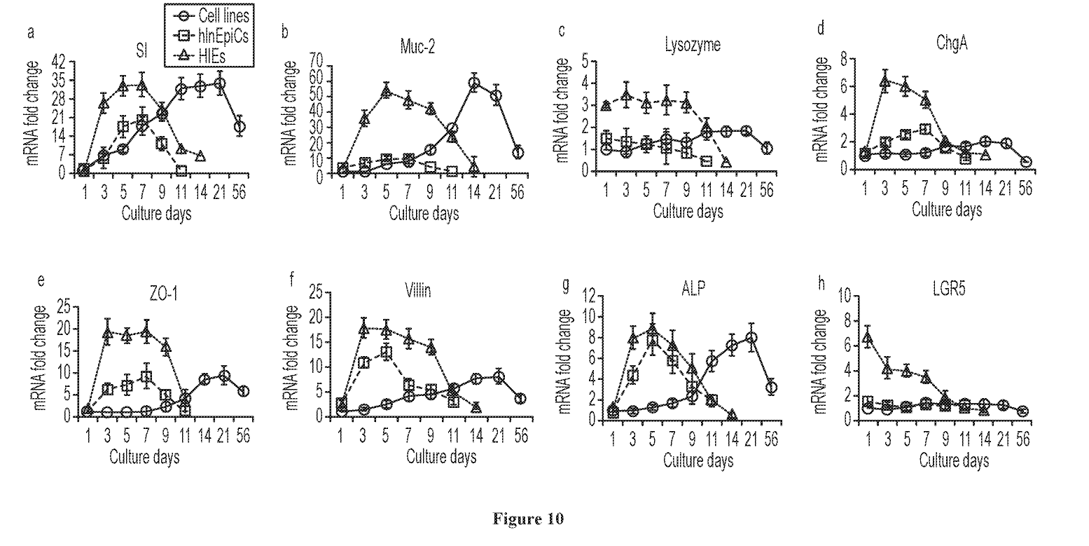

The oxygen concentration profiles were measured using a PC-controlled Microx TX3 oxygen meter (PreSens Precision Sensing GmbH) equipped with a needle-type housing fiber-optic oxygen sensor (NTH-PSt1-L5-TF-NS40/0.8-OIW, 140 μm fiber tapered to a 50 μm tip). Prior to use, a two-point calibration was performed according to the manufacturer's protocols with oxygen-free water (1% sodium sulfite, Sigma) corresponding to the 0% oxygen partial pressure and with air-saturated water corresponding to 100%. The needle probe was mounted on a custom-made micromanipulator capable of precisely positioning the measurement spot in the vertical direction. One complete turn of the screw knob resulted in 0.1 inch (2.5 mm) of travel. HIE-derived cells were cultured in 3D structures for 3 days post differentiation, hInEpiC-derived cells were cultured in 3D structures for 5 days post cell seeding, and cell line-derived cells were cultured in 3D structures for 15 days post cell seeding. Each of the 3D intestinal tissue scaffolds was then placed in an Eppendorf tube with its luminal direction oriented perpendicularly, and allowed to stabilize for 1 to 2 hours before taking measurements. In each step of probe advancement (0.05 inch/step), the oxygen tension reading was allowed to equilibrate for at least 5 minutes followed by data recording. At the end of each depth-profile measurement, the probe was retracted and the process was repeated 3 times for each sample. Five oxygen readings (30 sec/reading) were collected at each measurement position, subsequently averaged and plotted (FIG. 10). To ensure the comparability between different samples, all three profiles were determined on the same day (within 6 hours) using the same probe and calibration.

Co-Culture of Escherichia coli (E. coli) on 3D Silk Scaffolds:

The E. coli (BL23(DE3)), was used for infection experiments. Bacteria were grown overnight into stationary phase in 2xYT broth (LB, with 2× yeast extract and tryptone) at 37° C. with rotation. The bacterial cells were harvested at the mid-log phase of growth (O.D.600=0.6) by centrifugation (3,000×g, 10 min, 4° C.), washed with PBS and resuspended to an O.D.600 of 0.1 (˜10⁷ cells/mL) in Lysogeny broth (LB) medium at 37° C. with rotation overnight. Prior to bacterial inoculation, monolayers on scaffold lumens were washed with PBS, and cultured with fresh antibiotic-free medium supplemented with 5% inactivated fetal bovine serum for 24 hours. 3×10⁷ total CFUs were added to each scaffold.

Quantitative RT-PCR:

Intestinal epithelial cells on the luminal surface of scaffolds were detached with 0.25% trypsin-EDTA and a cell scraper. Total RNA was isolated using the Qiagen Mini mRNA Extraction kit. RNA was reverse-transcribed using High-Capacity cDNA Reverse Transcription Kit (Invitrogen, Carlsbad, Calif.) following the manufacturer's instructions. Six nanograms of cDNA were used for real-time PCR amplification for each well, using primer sequences shown in Table 2. For each gene tested we performed three experimental replicates and four biological replicates. Gene expression levels were normalized to the GAPDH mRNA level.

TABLE 2
qRT-PCR Primer List

| Gene | | |
|---|---|---|
| products | Forward | Reverse |
| GAPDH | GAAGGTGAAGGTCGGAG | GAAGATGGTGATGGGATT |
| | TC | TC |
| ZO-1 | CTGGTGAAATCCCGGAA | TTGCTGCCAAACTATCTTG |
| | AAATGA | TGA |
| E-caherin | ATCGGTTGTTCAATGCGT | CCTTCAGGATTTGGTACAT |
| | CC | GACA |
| Villin | CGGAAAGCACCCGTATG | CGTCCACCACGCCTACATA |
| | GAG | G |
| SI | TCCAGCTACTACTCGTGT | CCCTCTGTTGGGAATTGTT |
| | GAC | CTG |
| Chga | ACTCCGAGGAGATGAAC | CTTGGAGAGCGAGGTCTT |
| | GGA | GG |
| Lysozyme | CGCTACTGGTGTAATGAT | TTTGCACAAGCTACAGCAT |
| | GG | C |
| Muc-2 | TGCCTGGCCCTGTCTTTG | CAGCTCCAGCATGAGTGC |
| ALP | TACACGTCCATCCTGTAC | CTCGCTCTCATTCACGTCT |
| | GG | GG |
| Lgr-5 | GAGAAAGCATTTGTAGG | ATCTCCCAACAAACTGGAT |
| | CAAC | G |

PCR Array for the Antibacterial Response Genes:

A human antibacterial response RT² profile PCR array was performed as per the manufacturer's instructions (Qiagen, Valencia, Calif.). Total RNA was extracted from uninfected and infected HIE-derived, hInEpiC-derived and cell line-derived scaffolds respectively. cDNA was prepared as mentioned in Quantitative RT-PCR section. The cDNA was mixed with RT² qPCR master mix supplied by the manufacturer and real time PCR was performed in a 96-well plate format using Mx3000P qPCR System (Agilent Technologies, Santa Clara, Calif.). Data were analyzed using RT² Profiler PCR Array Data Analysis Software version 3.5. β-actin gene was used for normalization.

Statistical Analyses:

Data are presented as mean±SEM (n=3-5). A two tailed t-test was performed to compare means between two groups, and Analysis of Variance (ANOVA) was performed to compare means of multiple groups. P-values ≤0.05 were considered significant.

Results

The Establishment of HIE-Derived Primary Intestinal Epithelium:

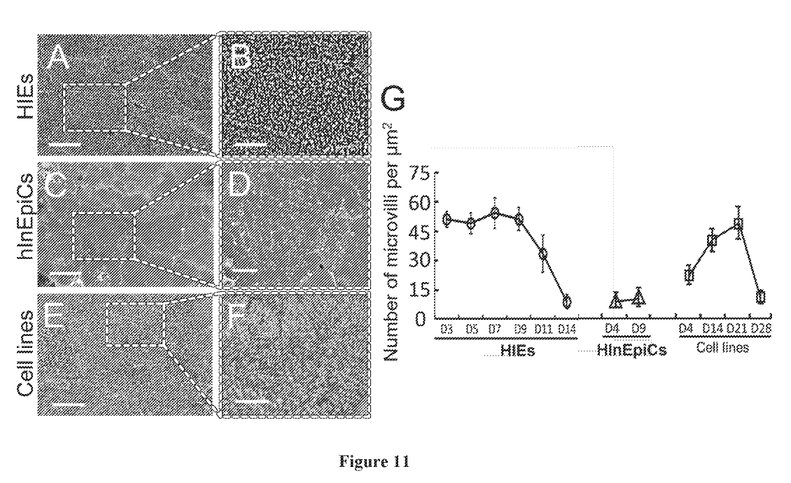

In the present study, we employed 3D hollow silk scaffold systems that our group previously developed for intestine engineering. As previously reported, this silk-based scaffold system consists of a hollow channel space (diameter, 2 mm) and a bulk space around the channel containing interconnected pores (FIG. 9d). We bioengineered the primary intestine model by cultivating HIE-derived primary epithelial cells on the luminal surface of silk scaffolds and primary human intestinal myofibroblasts (H-InMyoFibs) within the scaffold bulk space as feeder cells (FIG. 9a-d). After cell seeding, the HIE-derived scaffolds were maintained in growth medium overnight and then differentiation medium for up to 14 days. Three days after tissue differentiation, the primary small intestinal epithelial cells derived from HIEs formed confluent monolayers on the luminal surface of 3D silk scaffolds. The process of differentiation led to the formation of brush border with well-developed microvilli (FIG. 9e and FIG. 11), the presentence of apical ZO-1 tight junctions (FIG. 9f), and a polarized distribution of membrane components, such as digestive enzymes, ALP (FIG. 9g).

Identification of Four Major Epithelial Cell Populations:

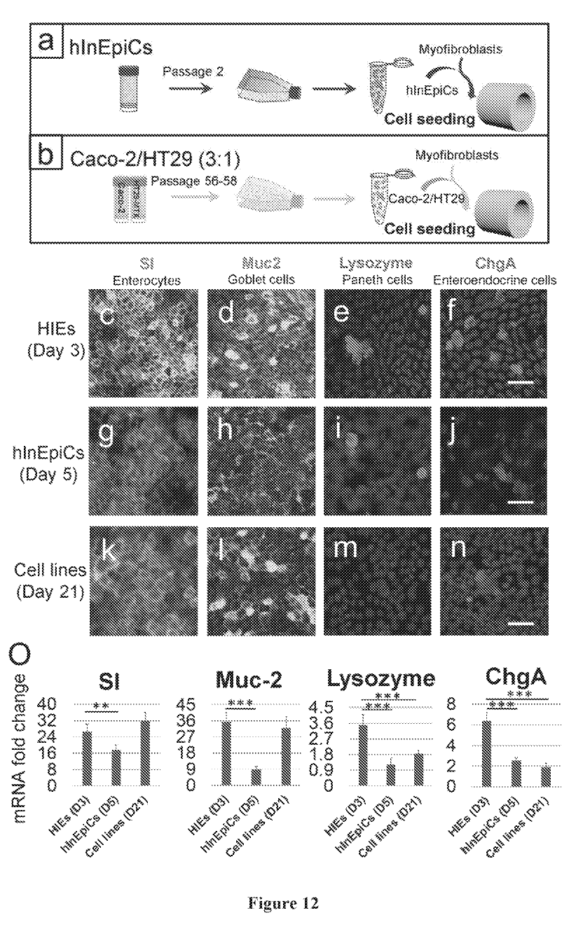

Native human small intestinal epithelium is populated with four major epithelial cell types: enterocytes, Goblet cells, enteroendocrine cells, and Paneth cells. Thus, we next aimed to identify the four cell populations from HIE-derived epithelium under differentiation on 3D silk scaffolds. Two other major cell sources for in vitro intestine engineering, Caco-2/HT29-MTX and hInEpiCs, seeded in the same scaffolds were used for the comparison (FIG. 12a, b). Antibodies for each cell population were used for immunostaining. Using confocal microscopy, in HIE-derived epithelium, enterocytes were identified by Sucrase-isomaltase (SI) (FIG. 12c), an enterocyte-specific, brush-border enzyme; Goblet cells by Mucin 2 (Muc2) (FIG. 12d), a mucin exclusively and abundantly expressed by goblet cells; Paneth cells by Lysozyme (FIG. 12e), specific marker for mature Paneth cells; and EECs by Chromogranin A (ChgA) (FIG. 120, a general cell surface markers for the enteroendocrine cells. Four markers were also observed in hInEpiC-derived epithelium (day 5 post cell seeding); however, the intensity of staining was relatively weaker. In cell line-derived epithelium (day 21 post cell seeding), the staining of Lysozyme and ChgA was not detectable, which means no Paneth cells and EECs present in the system. In addition to locating the protein expression of these markers by imaging, their gene expression in the epithelium grown in 3D constructs were also assessed by quantitative PCR. We found that mature HIE-derived epithelium (day 3) had the overall highest expression of these four marker transcripts than hInEpiC-derived (day 5) and cell line-derived epithelia (day 21) (FIG. 12o).

Intestine-Specific Gene Expression Analysis:

Typically, the maturity of the differentiated cells is evaluated by transcript levels of representative characteristic markers. To further characterize HIE-derived epithelial cell genotypes within the 3D scaffold culture, we performed mRNA expression analysis to directly quantify the gene expression levels of an extensive panel of known intestinal differentiation markers over time (FIG. 10). The markers included the four abovementioned epithelial cell markers (SI, Muc2, Lysozyme and ChgA), mature epithelium markers (ZO-1, Villi and ALP), and an intestinal stem cell marker, Lgr5. HIE-derived epithelium showed a significant upregulation (˜6-26 fold) of all marker genes after 3 days of cultivation in differentiation medium, with stable expression levels until around day 9 (FIG. 10). In contrast, hInEpiC-derived constructs only showed upregulated mRNA expression levels of SI, CghA, ZO-1, Villin, and ALP at day 5. The mRNA expression levels of the genes began to go down after day 7 (FIG. 10a, d, e, f, g). Cell line-derived constructs achieved stable expression of marker genes between days 14-21. Although the enterocyte marker (SI) and Goblet cell marker (Muc-2) were highly expressed in cell line-derived epithelium on 3D scaffolds (FIG. 10a, b), the Paneth cell marker (Lysozyme) and EEC marker (ChgA) were almost undetectable (FIG. 10c, d). Generally, HIE-derived and hInEpiC-derived epithelia survived for shorter terms in culture (˜9-12 days) than cell line-derived epithelium (˜8 weeks); however, differentiated HIE-derived epithelium on 3D scaffolds reached maturity earlier (˜3 days) than the hInEpiC-derived (˜5-7 days) and the cell line-derived (˜15-21) epithelia. Moreover, the overall expression levels of all markers from HIE-derived epithelium on 3D scaffolds were significantly higher than cell line-derived and hInEpiC-derived epithelia across all time points. The expression of Lgr5 transcript was only detectable in HIE-derived scaffolds and declined after differentiation (FIG. 10h).

Oxygen Profiles in the Scaffold Lumens:

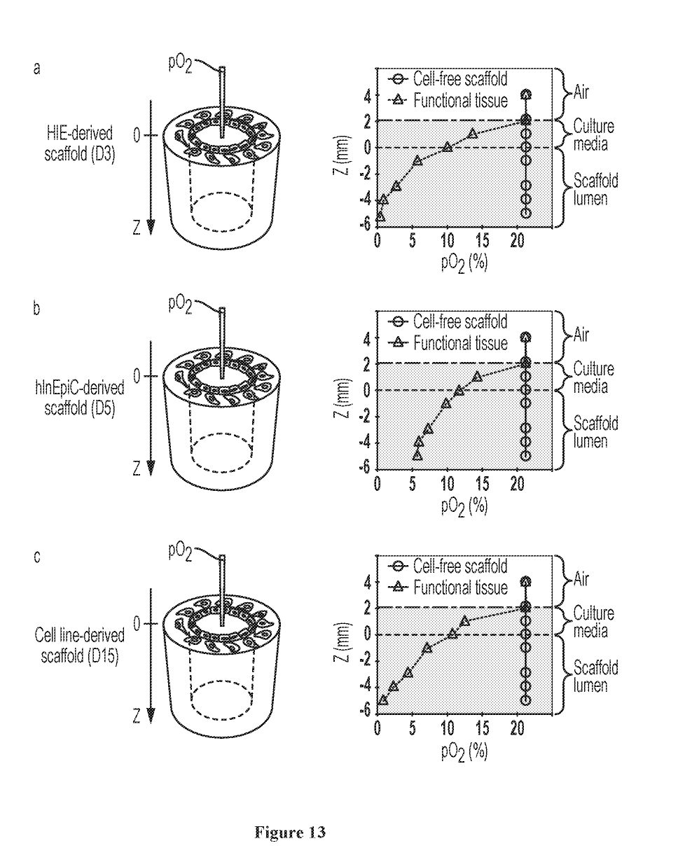

An important feature of our 3D scaffold system is the hollow channel compartment for epithelial cell growth. This hollow structure enables decreased oxygen levels which mimic in vivo conditions through oxygen consumption kinetics and metabolic activities of the cells in the lumen. In this study, we aimed to investigate whether HIE-derived cells grown in the lumen of the 3D scaffolds would also experience low oxygen tension. Similar to cell line-derived scaffolds (FIG. 13c), HIE-derived scaffolds also exhibited depth-graded oxygen profiles in the luminal direction (FIG. 13a). In the HIE-derived scaffolds, a region of microaerobic conditions (pO₂ between 5% and 1%) was detected at depths ranging from 2 to 5 mm into the scaffold lumen; a nanaerobic region (pO₂ ˜1%) was detected at the depth of 5 to 6 mm. However, in hInEpiC-derived scaffolds, the lowest pO₂ measured in the lumen was ˜6% (FIG. 13b).

Antibacterial Response to E. coli Infection:

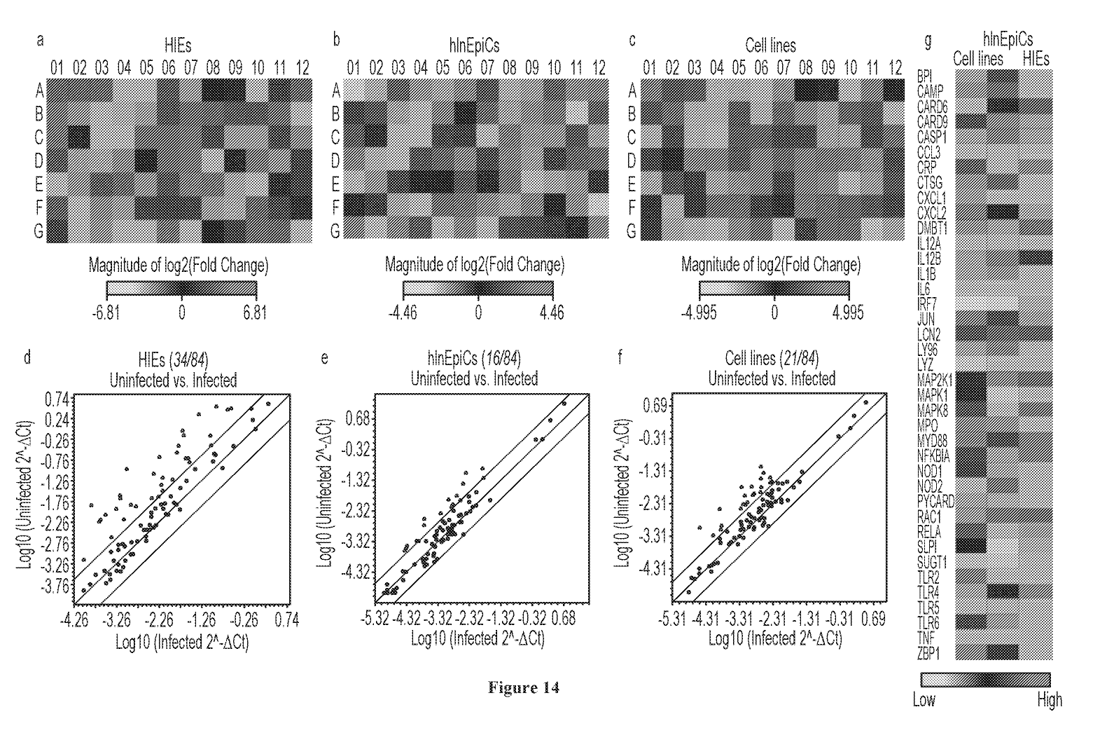

To determine the innate response of the intestinal epithelium against bacterial pathogens, we performed Human Antibacterial Response RT2 Profiler™ PCR arrays. Differentiated HIE-derived epithelium (3 days post differentiation), hInEpiC-derived epithelium (5 days in culture) and cell line-derived epithelium (15 days in culture) were separately incubated with a non-invasive strain of E. coli (BL23(DE3)). Gene expression profiles of epithelial cells from the three epithelial models were determined by the PCR array at 4 hours post inoculation, and compared with controls of each cell source without E. coli co-culture. The gene expression ratios between infected HIE-derived epithelium, infected hInEpiC-derived epithelium or infected cell line-derived epithelium and their corresponding uninfected control samples for all 84 genes were clustered and displayed in heat maps where individual elements of the plot are colored according to their standardized expression values (FIG. 14a-c; red squares: upregulated genes; green squares: downregulated genes). Compared to hInEpiC-derived and cell line-derived epithelia, HIE-derived epithelium displayed more upregulation after exposure to E. coli (more red squares). To more directly illustrate how these various source-derived epithelia on 3D scaffolds responded to the E. coli infection, the standardized expression values of all upregulated genes in FIG. 14a-c are displayed as a heat-map detailed in FIG. 14g for the cell line derived, hInEpiC-derived and HIE-derived epithelium samples, respectively (FIG. 14g; red squares: high expression; green squares: low expression). The dominant bright red color in HIE-derive epithelium indicated the enhanced antibacterial response of HIE-derived epithelium compared to hInEpiC-derived and cell line-derived epithelia. Based on gene selection criteria (P<0.05 and fold change ≥4), we identified 34 upregulated genes for HIE-derived epithelium, 16 upregulated genes for hInEpiC-derived epithelium, and 21 upregulated genes for cell line-derived epithelium (FIG. 14d-f). Amongst all of the induced genes, microbial sensors/bacterial pattern recognition receptors (LY96, TLR2, TLR4, TLR5, TLR6, CRP, DMBT1, IRF7 ZBP1) and proinflammatory cytokines/chemokines (CCL3, CXCL1, CXCL2, IL12A, IL12B, IL1B, IL6) were predominant, followed by inflammatory mediator genes (MYD88, NOD1, NOD2, RAC1, RELA, TNF), antimicrobial genes (BPI, CAMP, CTSG, LYZ, MPO, SLPI), downstream signal transduction genes (MAP2K1, MAPK1, MAPK8, JUN, NKB1A), and some inflammasome signaling genes (CASP1, PYCARD).

### Discussion/Conclusions

As described herein, the combination of stem cells and 3D biomaterial scaffolds has emerged as a promising strategy for tissue engineering. Stem cell-derived enteroids from human patients have become a valuable ex vivo model of normal human intestinal epithelia, allowing the indefinite establishment and propagation from normal nontumorigenic human specimens. In this Example, stem cell-derived spherical HIEs from the intestinal crypt were used to investigate the possibility of growing the HIE-derived primary epithelial cells in the 3D tubular silk scaffold system in vitro with H-InMyoFibs embedded in the system bulk for the tissue engineering of a 3D primary human intestinal epithelium. The results suggest that the 3D silk scaffold system supports a primary functional intestinal epithelium derived from HIEs. The resulting epithelial tissues formed in the 3D scaffolds consisted of multiple differentiated and undifferentiated stem cells found in human native intestine, expressed elevated levels of transcripts of intestinal markers, generated low oxygen tension in the lumen, and demonstrated a significant anti-bacterial response to bacterial infections.

In this Example, we adapted a previously published protocol for the isolation and differentiation of single stem cells from patient-derived enteroids. As shown, the single cells formed a confluent monolayer on the luminal surface of the 3D scaffolds. After culture in differentiation medium, four major native human intestinal epithelial cells were identified in the mature HIE-derived epithelium on 3D scaffolds by immunofluorescence analysis for protein expression (FIG. 12c-f) and qRT-PCR for expression of cell population-specific transcripts (FIG. 10o). The cells included the absorptive enterocyte cells, the mucus-producing Goblet cells, the hormone-secreting EECs, and the antimicrobial peptide secreting Paneth cells. The epithelium also developed mature epithelial markers, including ZO-1 tight junctions, dense microvilli with brush border, and ALP production (FIG. 9e-g). Additionally, the gene expression of crypt stem cell marker Lgr5 was also detected in the tissue, indicating the existence of intestinal stem cells in culture (FIG. 10h). These findings suggested that the 3D silk scaffold system provides an appropriate epithelial niche for the adhesion, proliferation and differentiation of intestinal stem cells. Silk protein as a scaffold has been fabricated to support wide variety of stem cells for different tissue engineering applications, such as cartilage, bone, adipose, etc. To our knowledge, this study is the first attempt at exploiting such systems for intestinal stem cell culture which permit cellular remodeling and tissue regeneration of a primary human intestinal epithelium in vitro.

Comparison of the gene expression of intestinal epithelial markers over time by qRT-PCR between HIE-derived, hInEpiC-derived and cell line-derived epithelia revealed distinctions between these cell lineages (FIG. 10). In the three epithelia, the differentiated HIE-derived epithelium was characterized by the highest biological complexity with the highest expression of major marker transcripts. By contrast, cell lines are of cancer cell origin and can only represent one cell population of human intestine. Interestingly, though hInEpiCs are primary cells directly isolated from human tissues and express most intestinal epithelial markers, the overall gene expression levels of hInEpiC-derived epithelium were found to be lower than that of the HIE-derived epithelium. This result is in contrast with findings where marker expression of primary intestinal epithelial cells and intestinal stem cell-derived epithelial cells were on the same order of magnitude. This inconsistency could be due to the source and passage of the primary cells used in the study. To obtain high enough cell numbers for scaffold seeding, we used cells from passage 2 after purchase from the vendor. Primary human cells tend to lose their genotype with passage in culture. With this consideration, the lower expression of intestinal marker genes in the hInEpiC-derived epithelium could be explained by the loss of cell genotype during passaging. These results indicate that human stem cell-derived enteroids may be the best choice for in vitro remodeling of human intestinal epithelium.

Low oxygen tension is critical for intestinal tissue function, as it is required for maintenance of a healthy gut microbial community. In vitro generation and dynamic control of oxygen gradients mimicking in vivo intestinal tissue, which vary from the anaerobic lumen across the epithelium into the highly vascularized sub-epithelium, has been a challenge for bioengineering and tissue regeneration.

In an effort to overcome this challenge, we developed a 3D tubular silk scaffold system for intestine engineering in which cells are seeded on the luminal surface, which provides stable access to the full range of oxygen conditions but without exposure to a low oxygenated cultivation atmosphere. In this Example, we take advantage of the luminal geometry to cultivate HIEs. Similar to cell line-derived intestinal models, the 3D in vitro HIE-derived intestinal models were also capable of reaching microaerobic, nanaerobic or anaerobic conditions in a standard CO₂ incubator (21% O₂, 5% CO₂, 37° C.) (FIG. 13). Under physiological conditions, the intestinal mucosa experiences frequent and wide fluctuations in blood perfusion and metabolism. For example, the villus oxygen tension in the murine small intestine is reported as being ˜2% under normal condition but decreases to ˜0.5% during glucose absorption. During acute or early stage gastrointestinal tract infections, pathogenic microorganisms and toxins, which enter the intestinal lumen and disrupt the mucous layer, trigger or exaggerate imbalances in tissue oxygen supply and demand. The bioengineered oxygen profiles and outcomes in vitro provide opportunities to study the role of oxygen concentrations in a wide variety of biological scenarios such as physiological stresses and pathological stimuli.

While the tissue characteristics of the HIE-derived primary epithelium on 3D scaffolds are very encouraging based on intestinal marker expression and low luminal oxygen profiles, it was unclear how capable these cells are of activating immune defenses when exposed to bacterial infections. To investigate this, we challenged the 3D intestine tissues with laboratory E. coli to determine if and how they respond to a well-known intestinal pathogen. Human intestines are constantly exposed to a vast number and diversity of bacteria. To cope with the substantial microbial threats, the intestinal epithelium uses defense mechanisms which involve the activation of a number of microbial recognition and innate immune pathways, the secretion of diverse proinflammatory cytokines/chemokines and antimicrobial proteins to kill or prevent the growth of bacteria in infected tissues. In this study, we demonstrated that HIE-derived epithelia exhibit significant antibacterial responses, as evidenced by the increased expression of genes with important roles in pathogen recognition and the activation of immune responses, including microbial sensor genes, cytokines, inflammatory mediator genes, downstream signal transduction genes, and inflammasome signaling genes (FIG. 14). Interestingly, many of these genes are activated in the intestinal tissues of IBD patients. For example, IBD patients have increased mRNA expression of Toll-like receptors, TLR2, TLR4 and TLR6, in the distal colon during colitis; CRP (C-reactive protein) is a clinical biomarker of IBD, as patients diagnosed with Crohn's disease and ulcerative colitis have elevated CRP; cytokines, such as IL-6, IL-12A, IL-12B, IL-1B and CXCL2, were upregulated in active IBD patients at diagnosis and during therapy; the enhancement of both NOD1 and NOD2 mRNAs was detected in tissue biopsies from IBD patients; the TNF serum level was significantly increased in IBD patients compared to healthy controls; expression of SLPI mRNA are higher in patients with ulcerative colitis than in healthy controls or patients with Crohn's disease. Strikingly, multiple upregulated genes identified in the infected HIE-derived epithelium, including TLR6, CRP, CXCL12, SLPI, were not changed or only slightly upregulated in the infected hInEpiC-derived and cell line-derived epithelia. It has been reported that infection with E. coli triggers an immune response that may cause uncontrolled inflammation that occurs in Crohn's disease and other types of IBD. The results presented here suggested that the HIE-derived primary 3D intestinal epithelium not only replicates many in vivo characteristics of the human intestine, but also closely reflects the human innate immune response to bacterial infection, which may permit the in vitro study of host-microbe-pathogen interplay and pathogenesis of IBD.

In conclusion, this Example demonstrates the possibility of growing human intestinal enteroid-derived primary epithelial cells in vitro in a biocompatible 3D tubular silk scaffold system. Since HIEs are intestinal stem cells-derived, they can differentiate into all relevant intestinal epithelial cell types (enterocytes, Goblet cells, Paneth cells and enteroendocrine cells) required to recreate a physiologically relevant system. Importantly, HIEs are directly isolated from native intestine tissues donated by individual patients, which allow this system to study patient-specific disease mechanisms and drug responses. Moreover, the 3D primary intestinal epithelium tissue model closely mimics natural human infection. This promising feature will provide the basis for acute and chronic studies of interactions between the mammalian cells, bacterial infectious agents and the study of antibiotic resistance.

### Example 5—Provided Compositions Including a Functional Nervous System

In this Example, certain provided compositions are exemplified that provide an innervated, 3D human intestinal model that can be cultured for months, provides relevant (structure and function), and is generated from human cells. This system is a major advance from previously known organoid cultures, which are limited due to necrosis, sustainability and lack of compartmentalization control, among other factors. Previously known intestinal systems for studying the enteric nervous system rely on in vivo murine and embryonic chick systems, and in vitro 2D cell culture, or organoid models, with little focus on adult cell interactions and microbiome crosstalk. In contrast, some embodiments of the present invention provide in vitro 3D human innervated intestinal tissues that encompasses both human induced neural stem cells (hiNSCs) differentiated into pertinent enteric nervous system neural cell types, as well as enterocyte-like (Caco-2) and goblet-like (HT29-MTX) cells that create the intestinal epithelial layer that is both robust and capable of long term culture. These tissue models elevate experimental options in technology to understand neural circuits controlling the intestine, and can offer insight into those communicating with the microbiome.

In this Example, unless otherwise specified, the materials and methods used were as follows:

### Materials & Methods

Cell Lines—

Human intestinal epithelial cells, Caco2 cell line obtained from ATCC (Rockville, Md.) and HT29-MTX obtained from the Public Health England Culture Collection (Salisbury, Great Britain) were cultured throughout the experiment. Both epithelial cell types were kept in DMEM with serum, 10% fetal bovine serum, 10 μg/mL human transferrin (Gibco), and 1% penicillin/streptomycin. Human Intestinal Myofibroblasts (H-InMyoFib) (Lonza) were cultured in SmGM™. Human Induced Neural Stem Cells (hiNSCs) were cultured in KO medium without fibroblast growth factor to induce differentiation toward neural linage. All cells, besides the hiNSCs, were kept in T175 culture flasks and maintained at 37° C. For hiNSCs, cells were maintained at 37° C. in 8 cm diameter petri dishes during differentiation.

Cell Seeding on Silk Scaffolds—

The human epithelial cells (Caco2 and HT29-MTX) were used to coat the inside of the scaffold lumen and the human intestinal myofibroblasts were used in the porous bulk space of the scaffold. Collagen solution with neural growth factor 50 ng/mL (NGF, R&D Systems), 80% rat-tail collagen, 10% 10×DMEM, and 10% 1×DMEM, was made in order to keep the human intestinal myofibroblasts in the spongy bulk space of the scaffolds and provide guidance for neural innervation into the scaffold bulk. Cells were left to populate the scaffolds for 1 week prior to hiNSC seeding. hiNSCs were allowed to differentiate toward neural linage for 1 week prior to seeding on scaffolds. hiNSCs were suspended in collagen solution without NGF, 80% rat tail collagen, 10% 10×DMEM, and 10% 1×DMEM, and coated on the outside of the mature scaffolds. The four conditions studied were intestinal cells only (Caco-2, HT29-MTX, and H-InMyoFib), hiNSCs only, co-culture (all listed cell types), and cell free scaffolds.

Extraction of Cells from Scaffolds—

Following culture, in order to collect data from the hiNSCs, and cell types localized to the bulk of the scaffold, all samples were rinsed three times with 1×DPBS and flash frozen in microcentrifuge tubes in a liquid nitrogen bath. Scaffolds were then stored at −80° C. These frozen scaffolds were then pulverized using the Bessman tissue pulvilizer. The weights of the pulverized samples were determined to assist with comparison of samples during analysis. Once cells were lysed from the freezing process and pulverized to assist with the release of cellular components from the silk scaffold an AllPrep, DNA, RNA, and protein extraction kit (Qiagen) was used to collect the DNA, RNA, and protein in each of the scaffold samples. For samples utilized for LC/MS, samples were purified and then run on one LC column utilizing 2 ionization models.

Formation of In Vitro Enteric Nervous System—

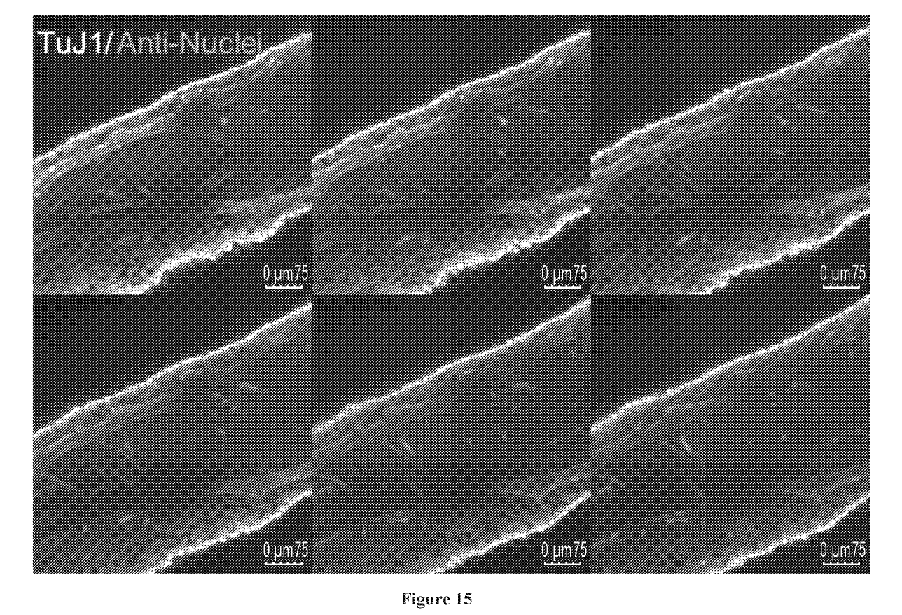

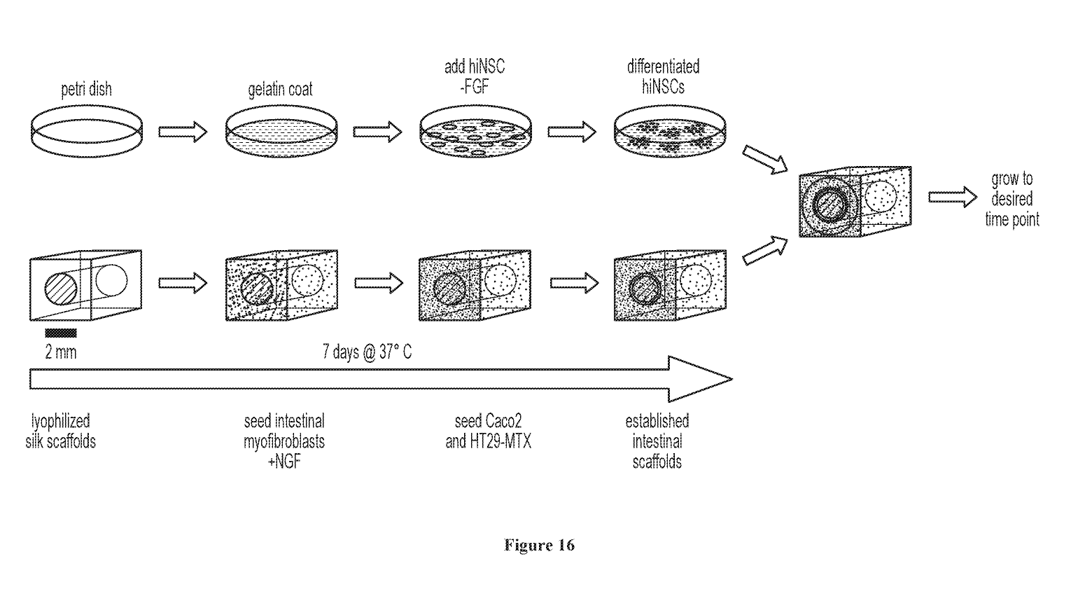

Previously, we developed an expandable and rapidly differentiating human induced neural stem cell (hiNSC) line that allowed for spontaneous differentiation toward neural linage (Cairns et al. 2016). In addition, we tested the capabilities of the hiNSCs to migrate into the intestine from the neural tube in order to show that the cell type is a viable neural source for the in vitro ENS. These cells expressed neural crest migration capabilities and were seen to have migrated to the intestine of D14 chicken embryos after injection into the neural tube at D3 (FIG. 15). In addition, we have generated a 3D intestine model capable of months-long sustained access to these intestinal functions in vitro that ensures a reliable ex vivo tissue system for studies in a broad context of human intestinal diseases and treatments (Chen et al., 2016). Although this in vitro system recapitulates many intestinal functions, including continuous mucus accumulation, that lack of an enteric nervous system limits the functionality. In order to generate an in vitro analog for the ENS we combined the hiNSCs and intestinal tissue models to develop a functional ENS in vitro model (FIG. 16).

Co-Culture with Intestinal Cells Enables ENS Differentiation—

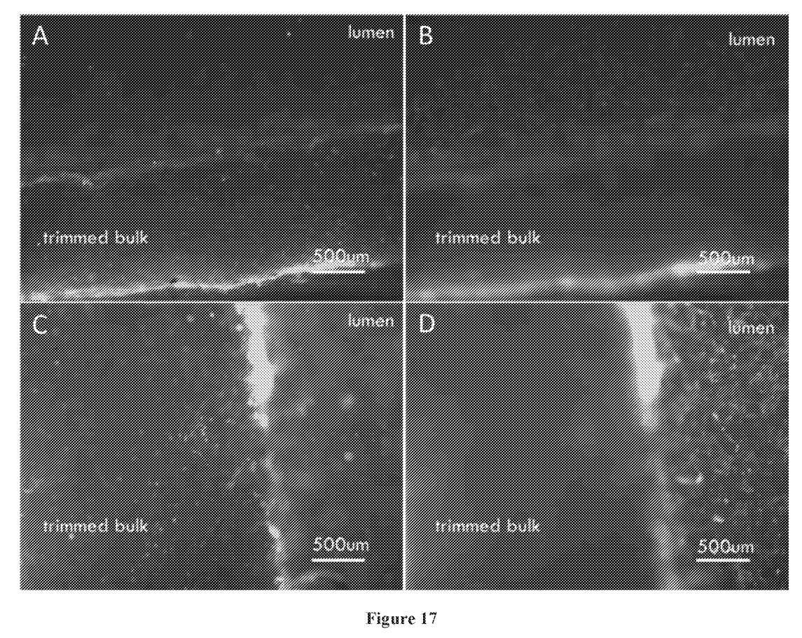

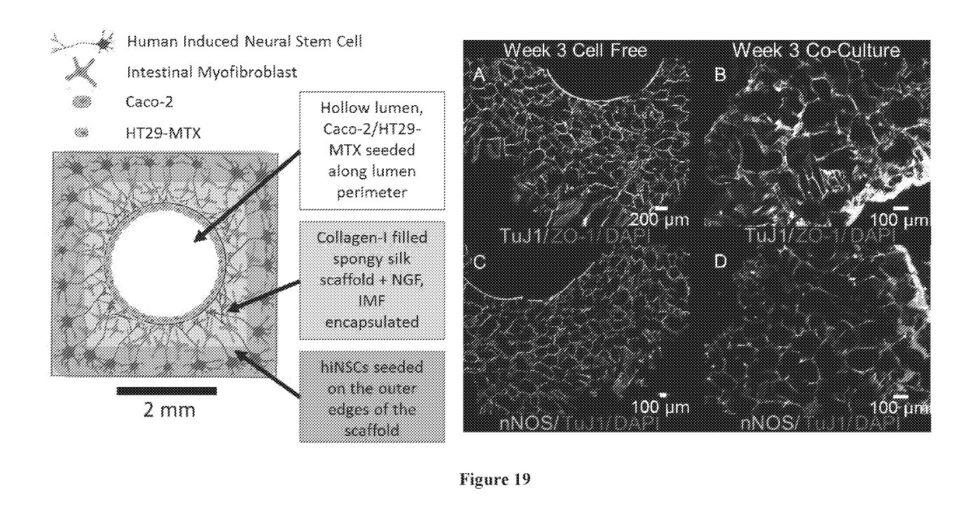

Cultures of in vitro ENS (ivENS) scaffolds indicate the capability to survive and metabolize out to day 21 (FIG. 17, 18), indicating the long term viability of experiments. Scaffolds were also assessed for penetration on hiNSCs toward the lumen utilizing histological cross sections of the scaffolds. Seeded hiNSCs are shown to migrate from their seeding location on the outside of the scaffold, indicated in FIG. 19 (panels B, D) by the dense population of TuJ1 positive cells, toward the lumen of the scaffold where epithelial cells were seeded.

A subset of intestinal neurons are responsible for smooth-muscle relaxation and the neuromodulatory capability is pertinent to normal intestinal function. These nNOS-expressing neurons can be found within the ivENS system, particularly toward the lumen of the scaffolds (FIG. 19, panel D). Previous work in the field has indicated that nNOS expression can only be seen following transplantation into host mice, thus the ability to incorporate nNOS expression into an all human in vitro allows for greater insight into the bidirectional communications between intestinal cells and the nervous system.

We claim:

1. A composition comprising a plurality of enterocytes;
a plurality of fibroblasts;
a plurality of Goblet cells;
a plurality of Paneth cells;
a plurality of enteroendocrine cells; and
a silk fibroin scaffold, wherein the composition exhibits one or more of tight junction formation, microvilli polarization, digestive enzyme secretion, and low oxygen tension.

2. The composition of claim 1, further comprising a plurality of nervous system cells.

3. The composition of claim 2, wherein the nervous system cells are human nervous system cells.

4. The composition of claim 2 or 3, wherein the nervous system cells are or comprise afferent nerve cells.

5. The composition of claim 2 or 3, wherein the nervous system cells are or comprise efferent nerve cells.

6. The composition of claim 2 or 3, wherein the nervous system cells comprise glial cells.

7. The composition of any one of claims 2-6, wherein the nervous system cells comprise neuronal nitric oxide synthase (nNOS)-expressing neurons.

8. The composition of any one of claims 2-7, wherein at least some of the plurality of nervous system cells provide functional innervation to at least some of the enterocytes, Paneth cells, enteroendocrine cells, and/or Goblet cells.

9. The composition of any one of the above claims, wherein digestive enzyme secretion is or comprises secretion of one or more of alkaline phosphatase, secretin, cholecystokinin, maltase, lactase, gastric inhibitory peptide, motilin, somatostatin, erepsin, and sucrase.

10. The composition of any one of the above claims wherein low oxygen tension means less than 5% pO₂.

11. The composition of any one of the above claims, wherein the composition is capable of initiating an antimicrobial response.

12. The composition of claim 11, wherein an antimicrobial response is or comprises upregulated gene and/or protein expression of one or more of lymphocyte antigen 96 (LY96), toll-like receptor-2 (TLR2), toll-like receptor-4 (TLR4), toll-like receptor-5 (TLR5), toll-like receptor-6 (TLR6), c-reactive protein (CRP), deleted in malignant brain tumors-1 (DMBT1), interferon regulatory factor-7 (IRF7), z-DNA-binding protein 1 (ZBP1), chemokine (C-C motif) ligand 3 (CCL3), C-X-C motif chemokine 1 (CXCL1), C-X-C motif chemokine 2 (CXCL2), interleukin-12 subunit alpha (IL12A), interleukin-12 subunit beta (IL12B), interleukin 1 beta (IL1B), interleukin 6 (IL6), myeloid differentiation primary response gene 88 (MYD88), nucleotide-binding oligomerization domain-containing protein 1 (NOD1), nucleotide-binding oligomerization domain-containing protein 2 (NOD2), Ras-related C3 botulinum toxin substrate 1 (RAC1), p65 (RELA), tumor necrosis factor (TNF), bactericidal permeability-increasing protein (BPI), cathelicidin (CAMP), cathepsin G (CTSG), lysozyme (LYZ), myeloperoxidase (MPO), secretory leukocyte protease inhibitor (SLPI), mitogen-activated protein kinase kinase 1 (MAP2K1), mitogen-activated protein kinase 1 (MAPK1), mitogen-activated protein kinase 8 (MAPK8), JUN, killer cell immunoglobulin-like receptor subunit a (NKB1A), caspace 1 (CASP1), and apoptosis-associated speck-like protein containing a CARD (PYCARD).

13. The composition of any one of the above claims, wherein the composition exhibits one or more of tight junction maintenance, maintenance of microvilli polarization, digestive enzyme secretion, and low oxygen tension for at least 10 days.

14. The composition of any one of the above claims, wherein the composition does not comprise any immortalized cells.

15. The composition of claim 14, wherein the composition does not comprise adenocarcinoma-based cells.

16. The composition of any one of the above claims, wherein all of the cells present in the composition are human cells.

17. The composition of any one of the above claims, wherein the silk fibroin scaffold is a film, a sponge, a tube, a mat, a gel, or any of the foregoing including a hollow channel.

18. The composition of claim 17, wherein the silk fibroin scaffold is porous.

19. The composition of any one of the above claims, further comprising at least one additional silk fibroin scaffold.

20. The composition of any one of the above claims, wherein at least one of the plurality of Enterocytes, Goblet cells, Paneth cells, and enteroendocrine cells originated from a patient.

21. The composition of any one of claims 2-20, further comprising an electrical device that is functionally connected to at least some of the plurality of nervous system cells.

22. The composition of claim 21, wherein the electrical device comprises at least one electrode.

23. The composition of claim 21 or 22, wherein the electrical device comprises silk fibroin.

24. A method comprising providing a silk fibroin scaffold;
associating a plurality of fibroblasts with the silk fibroin scaffold;
associating a plurality of intestinal stem cells with the silk fibroin scaffold;
differentiating the plurality of intestinal stem cells into two or more of enterocytes, Goblet cells, Paneth cells, and enteroendocrine cells to form an intestine-like composition.

25. The method of claim 24, further comprising associating a plurality of nervous system cells with the silk fibroin scaffold.

26. The method of claim 25, further comprising differentiating the plurality of nervous system cells into one or more of afferent nerve cells and efferent nerve cells.

27. The method of claim 25 or 26, wherein the plurality of nervous system cells are human nervous system cells.

28. The method of claim 2, wherein the intestinal stem cells are differentiated into three or more of enterocytes, Goblet cells, Paneth cells, and enteroendocrine cells.

29. The method of any one of claims 25-28, wherein the nervous system cells comprise neuronal nitric oxide synthase (nNOS)-expressing neurons.

30. The method of any one of claims 25-29, wherein at least some of the plurality of nervous system cells provide functional innervation to at least some of the enterocytes, Paneth cells, enteroendocrine cells, and/or Goblet cells

31. The method of any one of claims 24-30, wherein the intestine-like composition exhibits one or more of tight junction formation, microvilli polarization, digestive enzyme secretion, and low oxygen tension.

32. The method of claim 31, wherein digestive enzyme secretion is or comprises secretion of one or more of alkaline phosphatase, secretin, cholecystokinin, maltase, lactase, gastric inhibitory peptide, motilin, somatostatin, erepsin, and sucrase.

33. The method of claim 31 wherein low oxygen tension means less than 5% pO₂.

34. The method of claim 31 wherein low oxygen tension means less than 2% pO₂.

35. The method of any one of claims 24-34, wherein the intestine-like composition is capable of initiating an antimicrobial response.

36. The method of claim 35, wherein an antimicrobial response is or comprises upregulated gene and/or protein expression of one or more of lymphocyte antigen 96 (LY96), toll-like receptor-2 (TLR2), toll-like receptor-4 (TLR4), toll-like receptor-5 (TLR5), toll-like receptor-6 (TLR6), c-reactive protein (CRP), deleted in malignant brain tumors-1 (DMBT1), interferon regulatory factor-7 (IRF7), z-DNA-binding protein 1 (ZBP1), chemokine (C-C motif) ligand 3 (CCL3), C-X-C motif chemokine 1 (CXCL1), C-X-C motif chemokine 2 (CXCL2), interleukin-12 subunit alpha (IL12A), interleukin-12 subunit beta (IL12B), interleukin 1 beta (IL1B), interleukin 6 (IL6), myeloid differentiation primary response gene 88 (MYD88), nucleotide-binding oligomerization domain-containing protein 1 (NOD1), nucleotide-binding oligomerization domain-containing protein 2 (NOD2), Ras-related C3 botulinum toxin substrate 1 (RAC1), p65 (RELA), tumor necrosis factor (TNF), bactericidal permeability-increasing protein (BPI), cathelicidin (CAMP), cathepsin G (CTSG), lysozyme (LYZ), myeloperoxidase (MPO), secretory leukocyte protease inhibitor (SLPI), mitogen-activated protein kinase kinase 1 (MAP2K1), mitogen-activated protein kinase 1 (MAPK1), mitogen-activated protein kinase 8 (MAPK8), JUN, killer cell immunoglobulin-like receptor subunit a (NKB1A), caspace 1 (CASP1), and apoptosis-associated speck-like protein containing a CARD (PYCARD).

37. The method of any one of claims 24-36, wherein the intestine-like composition exhibits one or more of tight junction maintenance, maintenance of microvilli polarization, digestive enzyme secretion, and low oxygen tension for at least 10 days.

38. The method of any one of claims 24-37, wherein the intestine-like composition does not comprise any immortalized cells.

39. The method of claim 38, wherein the intestine-like composition does not comprise adenocarcinoma-based cells.

40. The method of any one of claims 24-39, wherein all of the cells present in the intestine-like composition are human cells.

41. The method of any one of claims 24-40, wherein the silk fibroin scaffold is a film, a sponge, a tube, a mat, a gel, or any of the foregoing including a hollow channel.

42. The method of claim 41, wherein the silk fibroin scaffold is porous.

43. The method of any one of claims 24-42, further comprising at least one additional silk fibroin scaffold.

44. The method of any one of claims 24-43, wherein at least one of the plurality of Enterocytes, Goblet cells, Paneth cells, and enteroendocrine cells originated from a patient.

45. The method of any one of claims 25-44, further comprising an electrical device that is functionally connected to at least some of the plurality of nervous system cells.

46. The method of claim 45, wherein the electrical device comprises at least one electrode.

47. The method of claim 45 or 46, wherein the electrical device comprises silk fibroin.

48. A method comprising
providing a composition according to claim 1;
exposing the composition to one or more therapeutic agents; and
characterizing the response of one or more of the enterocytes, Goblet cells, Paneth cells, and enteroendocrine cells to the one or more therapeutic agents.

49. The method of claim 48, wherein at least some of the enterocytes, Goblet cells, Paneth cells, and/or enteroendocrine cells exhibit one or more pathologic abnormalities as compared to similar cells from a healthy individual prior to the exposing step.

50. The method of claim 49, wherein the one or more pathologic abnormalities is indicative of, or correlated to, the presence of a disease.

51. The method of claim 50, wherein the disease is selected from the group consisting of inflammatory bowel syndrome, Celiac Disease, Crohn's disease, intestinal cancer, intestinal ulcer, ulcerative colitis, and diverticulitis.
